(19) United States
(12) Patent Application Publication
(10) Pub. No.: US 2025/0388983 A1
(43) Pub. Date: Dec. 25, 2025

(54) MULTIPLEX PANEL FOR UPPER RESPIRATORY PATHOGENS

(71) Applicant: LIFE TECHNOLOGIES CORPORATION, Carlsbad, CA (US)

(72) Inventors: Fangqi HU, Palo Alto, CA (US); Linda WONG, San Bruno, CA (US); Kara NORMAN, San Francisco, CA (US)

(73) Assignee: LIFE TECHNOLOGIES CORPORATION, Carlsbad, CA (US)

(21) Appl. No.: 18/879,481

(22) PCT Filed: Jun. 28, 2023
(86) PCT No.: PCT/US2023/069277

§ 371 Date: Dec. 27, 2024

Related U.S. Application Data
(60) Provisional application No. 63/367,215, filed on Jun. 28, 2022.

(51) Int. Cl. C12Q 1/70; C12Q 1/48; C12Q 1/686

(52) U.S. Cl. CPC C12Q 1/701; C12Q 1/48; C12Q 1/686; C12Y 207/07; C12Q 2600/16

(57) ABSTRACT

An in vitro method, composition and kit for determining the presence or absence of adenovirus, metapneumovirus, rhinovirus/enterovirus, and parainfluenza in a sample, including providing a reaction mixture containing the sample and at least one primer pair set. The primer pair set includes at least one primer pair A that specifically amplifies a portion of adenovirus genome; at least one primer pair B that specifically amplifies a portion of metapneumovirus genome; at least one primer pair C that specifically amplifies a portion of rhinovirus/enterovirus genome; and at least one primer pair D that specifically amplifies a portion of parainfluenza genome. The reaction mixture is subjected to reaction conditions suitable to amplify targeted nucleic acids, thereby generating at least one amplicon; wherein the presence or absence of at least one amplicon in the sample indicates the presence or absence of adenovirus, metapneumovirus, rhinovirus/enterovirus, and/or parainfluenza in the sample.

### CROSS-REFERENCE TO RELATED APPLICATIONS

This application claims priority to U.S. Provisional Patent Application No. 63/367,215, titled “Multiplex Panel for Upper Respiratory Pathogens,” filed Jun. 28, 2022, the entirety of which is incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### FIELD OF THE INVENTION

The invention relates to nucleic acid-based kits and in vitro methods for determining the presence or absence of respiratory pathogens in a sample, the human respiratory pathogens including the following: adenovirus, metapneumovirus, rhinovirus/enterovirus, and parainfluenza.

### BACKGROUND OF THE INVENTION

Due to their common presentation and ease of spread, the detection of respiratory infections represents a substantial share of the molecular diagnostics market. Renewed interest in this diagnostic realm has recently been driven by COVID-19 testing needs, but this field represents the most diverse set of needs across customer segments. Multiplex respiratory panels to detect more common viral pathogens such as human metapneumovirus, adenovirus, parainfluenza virus, and rhinovirus/enterovirus are needed to provide highly sensitive and specific IVD products with simple workflow, high throughput, and low cost.

Accordingly, there is a need to detect the above-mentioned pathogens in a single assay that would be sensitive, specific for the targets and not cross-reactive. The need is solved by the presently disclosed method that is directed to provide the detection of human metapneumovirus (MPV), adenovirus (Adeno), parainfluenza virus (PIV), and rhinovirus/enterovirus (Rhino/Entero) in a multiplex qPCR assay.

### SUMMARY OF THE INVENTION

Disclosed herein is an in vitro method for determining the presence or absence of at least one of an adenovirus, a metapneumovirus, a rhinovirus/enterovirus, and/or a parainfluenza in a sample, said method including (i.e., comprising) one or more of the following steps: (a) providing a reaction mixture that includes the sample and at least one primer pair set; wherein the at least one primer pair set includes at least one primer pair A that specifically amplifies a portion of adenovirus genome; at least one primer pair B that specifically amplifies a portion of metapneumovirus genome; at least one primer pair C that specifically amplifies a portion of rhinovirus/enterovirus genome; and at least one primer pair D that specifically amplifies a portion of parainfluenza genome; (b) subjecting the reaction mixture to reaction conditions suitable to amplify targeted nucleic acids, thereby generating at least one amplicon: wherein the presence or absence of at least one amplicon in the sample indicates the presence or absence of adenovirus, metapneumovirus, rhinovirus/enterovirus, and/or parainfluenza in the sample.

In an exemplary embodiment, the generating of the at least one amplicon includes subjecting the reaction mixture to polymerase chain reaction (PCR) conditions.

In an exemplary embodiment, the amplicon produced from using the primer pair A includes a sequence selected from the sequences specific for adenovirus in the “Probe” column of FIG. 1, the amplicon produced from using the primer pair B includes a sequence selected from the sequences specific for metapneumovirus in the “Probe” column of FIG. 1, the amplicon produced from using the primer pair C includes a sequence selected from the sequences specific for rhinovirus/enterovirus in the “Probe” column of FIG. 1, and/or the amplicon produced from using the primer pair D includes a sequence selected from the sequences specific for parainfluenza in the “Probe” column of FIG. 1.

In an exemplary embodiment, the reaction mixture contains probes specific for the amplicons generated in step (b).

In an exemplary embodiment, the reaction mixture further includes probes specific for amplicons generated using a specific forward and reverse primer pair, and the probes can be selected from any of the probe sequences provided in FIG. 1.

In exemplary embodiments, the primer pair set includes the following sequences: a primer pair A that includes at least one forward and reverse primer pair selected from primer pair sequences specific for adenovirus (i.e., Adeno) in FIG. 1, a primer pair B that includes at least one forward and reverse primer pair selected from the sequences specific for metapneumovirus (i.e., MPV) in FIG. 1, a primer pair C that includes at least one forward and reverse primer pair selected from the sequences specific for rhinovirus/enterovirus (i.e., Rhino/Entero) in FIG. 1, and a primer pair D that includes at least one forward and reverse primer pair selected from the sequences specific for parainfluenza (i.e., PIV) in FIG. 1.

In an exemplary embodiment, the reaction mixture further includes probes that detect amplicons produced from primer pair A, primer pair B, primer pair C and primer pair D, said probes having a sequence in FIG. 1 specific for detecting the amplicons.

In an exemplary embodiment, primer pair A includes at least one forward and reverse primer pair selected from the primer pair sequences in Table 1; primer pair B includes at least one forward and reverse primer pair selected from the primer pair sequences in Table 2; primer pair C includes at least one forward and reverse primer pair selected from the primer pair sequences in Table 3; and/or primer pair D includes at least one forward and reverse primer pair selected from the primer pair sequences in Table 4.

In an exemplary embodiment, the reaction mixture includes an additional primer pair set. The additional primer pair set includes an additional primer pair A including at least one forward and reverse primer pair sequence in Table 1; an additional primer pair B including at least one forward and reverse primer pair sequence in Table 2; an additional primer pair C including at least one forward and reverse primer pair sequence in Table 3; and/or an additional primer pair D including at least one forward and reverse primer pair sequence in Table 4.

In an exemplary embodiment, the reaction mixture further includes a primer pair E and, optionally, a probe, that specifically amplify at least a portion of the human RNase P gene.

In an exemplary embodiment, the primer pair E includes a forward and reverse primer pair sequence in Table 5 and the probe includes a probe sequence in Table 5.

In an exemplary embodiment, the probes each contain a fluorescent reporter.

In an exemplary embodiment, the probes each contain a quencher.

In an exemplary embodiment, each of the probes is labeled at or near the 5′ end with a dye selected from Alexa Fluor™, ABY™, VIC™, JUN™, and FAM™.

In example embodiments, each of the probes is labeled at the 3′ end with a quencher selected from QSY™, MGBNFQ, BHQ™, and DFQ.

In an exemplary embodiment, the probes specific for amplicons generated using at least one primer pair A are labeled with Alexa Fluor™, the probes specific for amplicons generated using at least one primer pair B are labeled with ABY™, the probes specific for amplicons generated using at least one primer pair C are labeled with VIC™, the probes specific for amplicons generated using at least one primer pair D are labeled with FAM™.

In an exemplary embodiment, the probes are specific for amplicons generated using at least one primer pair E are labeled with JUN™.

In an exemplary embodiment, the probe that specifically amplifies the at least a portion of the human RNase P gene is labeled with JUN™.

Disclosed herein is also a composition for determining the presence or absence of adenovirus, metapneumovirus, rhinovirus/enterovirus, and/or parainfluenza in a sample, the composition including one or more of the following: at least one primer pair set, wherein the at least one primer pair set includes; at least one primer pair A that specifically amplifies a portion of an adenovirus genome; at least one primer pair B that specifically amplifies a portion of a metapneumovirus genome; at least one primer pair C that specifically amplifies a portion of a rhinovirus/enterovirus genome; and at least one primer pair D that specifically amplifies a portion of a parainfluenza genome.

In an exemplary embodiment, the primer pair set includes the following sequences: a primer pair A that includes at least one forward and reverse primer pair selected from the primer pair sequences specific for adenovirus in FIG. 1, a primer pair B that includes at least one forward and reverse primer pair selected from the primer pair sequences specific for metapneumovirus in FIG. 1, a primer pair C that includes at least one forward and reverse primer pair selected from the primer pair sequences specific for rhinovirus/enterovirus in FIG. 1, and/or a primer pair D that includes at least one forward and reverse primer pair selected from the primer pair sequences specific for parainfluenza in FIG. 1.

In exemplary embodiments, the composition further includes probes specific for the amplicons produced using the primer pair A, the primer pair B, the primer pair C, and/or the primer pair D.

In an exemplary embodiment, the probes include a probe sequence provided in FIG. 1 that is specific for the amplicons produced using the at least one primer pair A, the at least one primer pair B, the at least one primer pair C, and the at least one primer pair D.

In an exemplary embodiment, the composition further includes a polymerase, a buffer, and nucleotides.

In an exemplary embodiment, the composition further includes a sample.

In an exemplary embodiment, the composition further includes a primer pair E and, optionally, at least one probe, that specifically amplify at least a portion of the human RNase P gene.

In an exemplary embodiment, the primer pair E includes a forward and reverse primer pair selected from Table 5 and at least one probe having a sequence selected from the probe sequences in Table 8.

In an exemplary embodiment, the at least one probe includes a fluorescent reporter.

In an exemplary embodiment, the at least one probe includes a quencher.

In an exemplary embodiment, the at least one probe is labeled at or near the 5′ end with a dye selected from Alexa Fluor™, ABY™, VIC™, JUN™, and FAM™.

In an exemplary embodiment, the at least one probe is labeled at the 3′ end with a quencher selected from QSY™, MGBNFQ, BHQ™, and DFQ.

In an exemplary embodiment, the probes specific for amplicons generated using at least one primer pair A are labeled with Alexa Fluor™, the probes specific for amplicons generated using at least one primer pair B are labeled with ABY™, the probes specific for amplicons generated using at least one primer pair C are labeled with VIC™, the probes specific for amplicons generated using at least one primer pair D are labeled with FAM™.

In an exemplary embodiment, the probes specific for amplicons generated using at least one primer pair E are labeled with JUN™.

In an exemplary embodiment, the probe specific for amplifying at least a portion of the human RNase P gene is labeled with JUN™.

Disclosed herein is also a kit for determining the presence or absence of adenovirus, metapneumovirus, rhinovirus/enterovirus, and parainfluenza in a sample, the kit including a composition in accordance with any of the exemplary embodiments described above.

In an exemplary embodiment, the kit further includes a primer pair E and, optionally, a probe, that specifically amplify at least a portion of the human RNase P gene.

In an exemplary embodiment, the primer pair E includes a forward and reverse primer pair selected from Table 5 and the probe includes a probe sequence selected from Table 5.

In an exemplary embodiment, the composition or kit includes at least one primer pair set and probe set provided any of the following tables: Table 6, Table 7, Table 8, Table 9, Table 10 and/or Table 11.

### BRIEF DESCRIPTION OF THE DRAWINGS

FIG. 1 provides exemplary forward and reverse primer pairs, together with a probe that is used with that primer pair (that is, a primer pair/probe set), as well as, the virus (or gene, in case of RNase P) that is detected with the primer pair/probe set (as can be determined by the column “virus or gene”). “Primer pair No.” denotes different primer pair/probe sets that can be used for detection of a particular virus.

### DETAILED DESCRIPTION

All publications and patent applications cited herein, as well as the Appendices attached hereto, are incorporated by reference in their entirety for all purposes to the same extent as if each individual Appendix, publication or patent application were specifically and individually indicated to be so incorporated by reference. Although the Appendices attached hereto may include particular examples that reference specific target nucleic acids, formulations, and process steps, it will be understood that these examples may be modified by using any of the formulations, components, and/or process steps described elsewhere herein, including by using any of the primers and/or probes described herein. Further, although the present disclosure has been described in some detail by way of illustration and example for purposes of clarity and understanding, it will be apparent that certain changes and modifications may be practiced within the scope of the spirit and substance of this disclosure and of the appended claims.

There are common viral pathogens that can cause upper and/or lower respiratory tract infections with similar clinical presentations. An assay that can identify these respiratory viruses is desirable. Here we describe a method, composition and kit for simultaneous detection of four common respiratory pathogens capable of causing upper respiratory tract infections and community acquired pneumonia: human rhinovirus/enterovirus (HRV/HEV), Adenovirus, Metapneumovirus (hMPV) and Parainfluenza viruses (HPIV types 1, 2, 3 and 4) via a multiplex RT-qPCR assay or multiplex dPCR assay. These multiplex panels include an RNase P assay as an internal control (IC). The RNase P gene is a reference human gene that serves as an endogenous marker, indicating successful and sufficient sample collection. The probes that target genomic regions of different pathogens utilize four distinct fluorophores, while a fifth fluorophore is designated for RNase P. Therefore, also a 5-plex qPCR assay is provided herein. The qPCR assay for detecting the respiratory viruses and/or RNase P can take place in a 1-well reaction.

When an exemplary “embodiment” or a particular “assay” is described herein, it will be understood that the features of the embodiment may be applicable to a composition (e.g., the particular physical components of an assay such as primers and/or probes), a kit (e.g., primers and/or probes and additional buffers, reagents, etc.), or a method (e.g., a process for detecting target nucleic acids) as appropriate. For simplicity, many embodiments are presented by describing “assays”, but it will be understood that the associated methods of using the assays are also intended to form part of this disclosure.

A method for determining the presence or absence of adenovirus, metapneumovirus, rhinovirus/enterovirus, and/or parainfluenza in a sample, said method including the steps of: (a) providing a reaction mixture including the sample and at least one primer pair set; wherein at least one the primer pair set includes; at least one primer pair A that specifically amplifies a portion of an adenovirus genome; at least one primer pair B that specifically amplifies a portion of a metapneumovirus genome; at least one primer pair C that specifically amplifies a portion of a rhinovirus/enterovirus genome; and at least one primer pair D that specifically amplifies a portion of a parainfluenza genome; (b) subjecting the reaction mixture to reaction conditions suitable to amplify targeted nucleic acids, thereby generating at least one amplicon; wherein the presence or absence of the at least one amplicon in the sample indicates the presence or absence of an adenovirus, a metapneumovirus, a rhinovirus/enterovirus, and/or a parainfluenza in the sample.

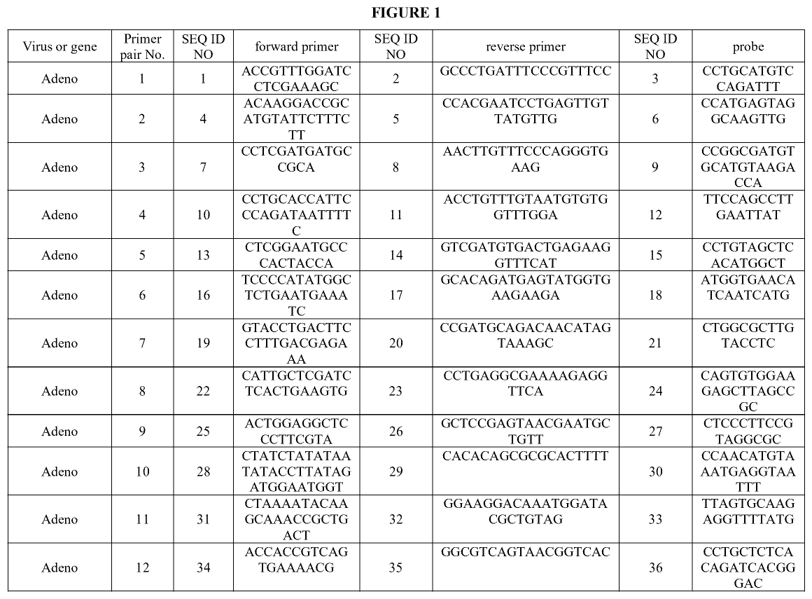

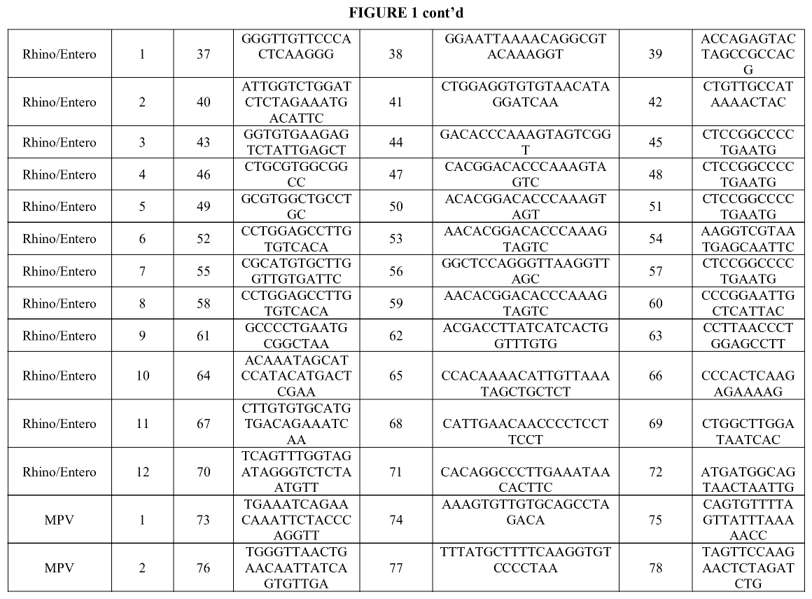

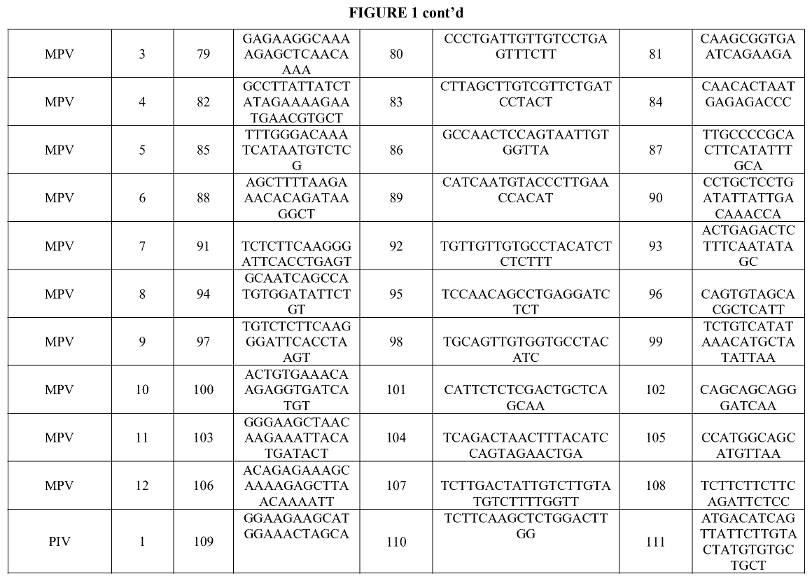

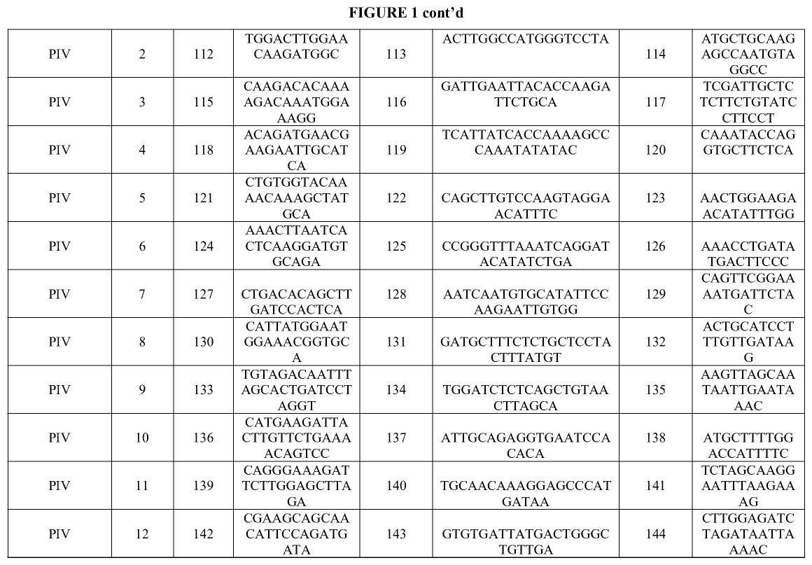

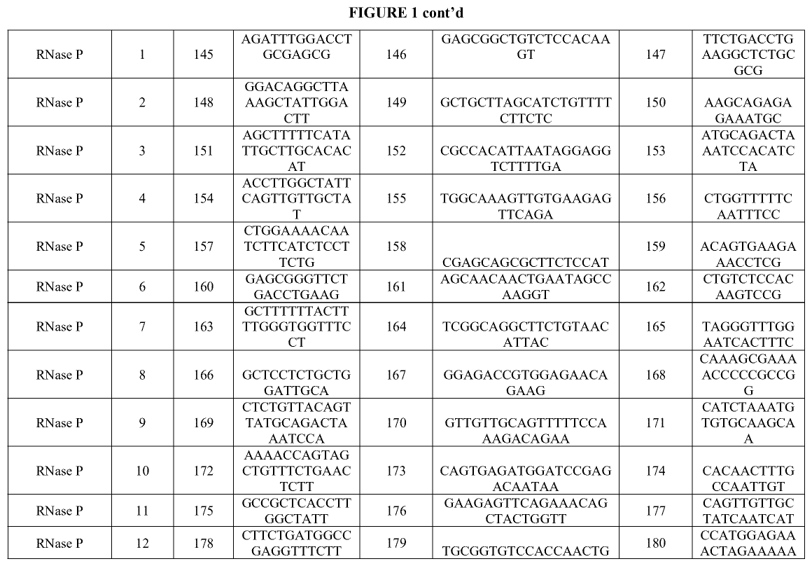

In exemplary embodiments, the primer pair set includes at least one primer pair A, at least one primer pair B, at least one primer pair C and at least one primer pair D. In exemplary embodiments, the primer pair set further includes at least one primer pair E. In exemplary embodiments, the primer pairs are used with corresponding probes as set out in FIG. 1. In exemplary embodiments, the primer pair set includes at least two primer pairs A, at least two primer pairs B, at least two primer pairs C and/or at least two primer pairs D.

In exemplary embodiments, the primer pair A includes at least one of the primer pairs in Table 1, in a combination with a corresponding probe sequence in Table 1:

TABLE 1

| | Primer | | | |
|---|---|---|---|---|
| virus | pair No. | forward primer | reverse primer | probe |
| Adeno | A1 | ACCGTTTGGATCCT | GCCCTGATTTCCCGTTT | CCTGCATGTCCA |
| | | CGAAAGC | CC | GATTT |
| Adeno | A2 | ACAAGGACCGCAT | CCACGAATCCTGAGTTG | CCATGAGTAGGC |
| | | GTATTCTTTCTT | TTATGTTG | AAGTTG |
| Adeno | A3 | CCTCGATGATGCCG | AACTTGTTTCCCAGGGT | CCGGCGATGTGC |
| | | CA | GAAG | ATGTAAGACCA |
| Adeno | A4 | CCTGCACCATTCCC | ACCTGTTTGTAATGTGT | TTCCAGCCTTGA |
| | | AGATAATTTTC | GGTTTGGA | ATTAT |
| Adeno | A5 | CTCGGAATGCCCAC | GTCGATGTGACTGAGA | CCTGTAGCTCAC |
| | | TACCA | AGGTTTCAT | ATGGCT |
| Adeno | A6 | TCCCCATATGGCTC | GCACAGATGAGTATGG | ATGGTGAACATC |
| | | TGAATGAAATC | TGAAGAAGA | AATCATG |
| Adeno | A7 | GTACCTGACTTCCT | CCGATGCAGACAACAT | CTGGCGCTTGTA |
| | | TTGACGAGAAA | AGTAAAGC | CCTC |
| Adeno | A8 | CATTGCTCGATCTC | CCTGAGGCGAAAAGAG | CAGTGTGGAAGA |
| | | ACTGAAGTG | GTTCA | GCTTAGCCGC |
| Adeno | A9 | ACTGGAGGCTCCCT | GCTCCGAGTAACGAAT | CTCCCTTCCGTAG |
| | | TCGTA | GCTGTT | GCGC |
| Adeno | A10 | CTATCTATATAATA | CACACAGCGCGCACTTT | CCAACATGTAAA |
| | | TACCTTATAGATGG | T | TGAGGTAATTT |
| | | AATGGT | | |
| Adeno | A11 | CTAAAATACAAGCA | GGAAGGACAAATGGAT | TTAGTGCAAGAG |
| | | AACCGCTGACT | ACGCTGTAG | GTTTTATG |
| Adeno | A12 | ACCACCGTCAGTGA | GGCGTCAGTAACGGTC | CCTGCTCTCACA |
| | | AAACG | AC | GATCACGGGAC |

In exemplary embodiments, the primer pair A includes at least two of the primer pairs as provided in Table 1, in a combination with the corresponding probe sequences in Table 1.

In exemplary embodiments, the primer pair B includes at least one of the primer pairs in Table 2, in a combination with a corresponding probe sequence in Table 2:

TABLE 2

| | Primer | | | |
|---|---|---|---|---|
| virus | pair No. | forward primer | reverse primer | probe |
| MPV | B1 | TGAAATCAGAAC | AAAGTGTTGTGCAGCCTA | CAGTGTTTTAGTT |
| | | AAATTCTACCCA | GACA | ATTTAAAAACC |
| | | GGTT | | |
| MPV | B2 | TGGGTTAACTGA | TTTATGCTTTTCAAGGTG | TAGTTCCAAGAAC |
| | | ACAATTATCAGT | TCCCCTAA | TCTAGATCTG |
| | | GTTGA | | |
| MPV | B3 | GAGAAGGCAAAA | CCCTGATTGTTGTCCTGA | CAAGCGGTGAATC |
| | | GAGCTCAACAAA | GTTTCTT | AGAAGA |
| | | A | | |
| MPV | B4 | GCCTTATTATCTA | CTTAGCTTGTCGTTCTGA | CAACACTAATGAG |
| | | TAGAAAAGAATG | TCCTACT | AGACCC |
| | | AACGTGCT | | |
| MPV | B5 | TTTGGGACAAAT | GCCAACTCCAGTAATTGT | TTGCCCCGCACTT |
| | | CATAATGTCTCG | GGTTA | CATATTTGCA |
| MPV | B6 | AGCTTTTAAGAA | CATCAATGTACCCTTGAA | CCTGCTCCTGATA |
| | | ACACAGATAAGG | CCACAT | TTATTGACAAACC |
| | | CT | | A |
| MPV | B7 | TCTCTTCAAGGG | TGTTGTTGTGCCTACATC | ACTGAGACTCTTT |
| | | ATTCACCTGAGT | TCTCTTT | CAATATAGC |
| MPV | B8 | GCAATCAGCCAT | TCCAACAGCCTGAGGAT | CAGTGTAGCACGC |
| | | GTGGATATTCTGT | CTCT | TCATT |
| MPV | B9 | TGTCTCTTCAAGG | TGCAGTTGTGGTGCCTAC | TCTGTCATATAAA |
| | | GATTCACCTAAG | ATC | CATGCTATATTAA |
| | | T | | |
| MPV | B10 | ACTGTGAAACAA | CATTCTCTCGACTGCTCA | CAGCAGCAGGGA |
| | | GAGGTGATCATG | GCAA | TCAA |
| | | T | | |
| MPV | B11 | GGGAAGCTAACA | TCAGACTAACTTTACATC | CCATGGCAGCATG |
| | | AGAAATTACATG | CAGTAGAACTGA | TTAA |
| | | ATACT | | |
| MPV | B12 | ACAGAGAAAGCA | TCTTGACTATTGTCTTGT | TCTTCTTCTTCAG |
| | | AAAGAGCTTAAC | ATGTCTTTTGGTT | ATTCTCC |
| | | AAAATT | | |

In exemplary embodiments, the primer pair B includes at least two of the primer pairs provided in Table 2, in a combination with the corresponding probe sequences in Table 2.

In exemplary embodiments, the primer pair C includes at least one of the primer pairs in Table 3, in a combination with a corresponding probe sequence in Table 3:

TABLE 3

| | Primer | | | |
|---|---|---|---|---|
| virus | pair No. | forward primer | reverse primer | probe |
| Rhino/ | C1 | GGGTTGTTCCCACTC | GGAATTAAAACAGGCGT | ACCAGAGTACT |
| Entero | | AAGGG | ACAAAGGT | AGCCGCCACG |
| Rhino/ | C2 | ATTGGTCTGGATCTCT | CTGGAGGTGTGTAACAT | CTGTTGCCATA |
| Entero | | AGAAATGACATTC | AGGATCAA | AAACTAC |
| Rhino/ | C3 | GGTGTGAAGAGTCTA | GACACCCAAAGTAGTCG | CTCCGGCCCCT |
| Entero | | TTGAGCT | GT | GAATG |
| Rhino/ | C4 | CTGCGTGGCGGCC | CACGGACACCCAAAGTA | CTCCGGCCCCT |
| Entero | | | GTC | GAATG |
| Rhino/ | C5 | GCGTGGCTGCCTGC | ACACGGACACCCAAAGT | CTCCGGCCCCT |
| Entero | | | AGT | GAATG |
| Rhino/ | C6 | CCTGGAGCCTTGTGT | AACACGGACACCCAAAG | AAGGTCGTAAT |
| Entero | | CACA | TAGTC | GAGCAATTC |
| Rhino/ | C7 | CGCATGTGCTTGGTT | GGCTCCAGGGTTAAGGTT | CTCCGGCCCCT |
| Entero | | GTGATTC | AGC | GAATG |
| Rhino/ | C8 | CCTGGAGCCTTGTGT | AACACGGACACCCAAAG | CCCGGAATTGC |
| Entero | | CACA | TAGTC | TCATTAC |
| Rhino/ | C9 | GCCCCTGAATGCGGC | ACGACCTTATCATCACTG | CCTTAACCCTG |
| Entero | | TAA | GTTTGTG | GAGCCTT |
| Rhino/ | C10 | ACAAATAGCATCCAT | CCACAAAACATTGTTAA | CCCACTCAAGA |
| Entero | | ACATGACTCGAA | ATAGCTGCTCT | GAAAAG |
| Rhino/ | C11 | CTTGTGTGCATGTGA | CATTGAACAACCCCTCCT | CTGGCTTGGAT |
| Entero | | CAGAAATCAA | TCCT | AATCAC |
| Rhino/ | C12 | TCAGTTTGGTAGATA | CACAGGCCCTTGAAATA | ATGATGGCAGT |
| Entero | | GGGTCTCTAATGTT | ACACTTC | AACTAATTG |

In exemplary embodiments, the primer pair C includes at least two of the primer pairs as provided in Table 3, in a combination with the corresponding probe sequences in Table 3.

In exemplary embodiments, the primer pair D includes at least one of the primer pairs in Table 4, in a combination with a corresponding probe sequence in Table

TABLE 4

| | Primer | | | |
|---|---|---|---|---|
| virus | pair No. | forward primer | reverse primer | probe |
| PIV | D1 | GGAAGAAGCAT | TCTTCAAGCTCTGGACT | ATGACATCAGTTATTCTT |
| | | GGAAACTAGCA | TGG | GTACTATGTGTGCTGCT |
| PIV | D2 | TGGACTTGGAA | ACTTGGCCATGGGTCCT | ATGCTGCAAGAGCCAAT |
| | | CAAGATGGC | A | GTAGGCC |
| PIV | D3 | CAAGACACAAA | GATTGAATTACACCAA | TCGATTGCTCTCTTCTGT |
| | | AGACAAATGGA | GATTCTGCA | ATCCTTCCT |
| | | AAGG | | |
| PIV | D4 | ACAGATGAACG | TCATTATCACCAAAAG | CAAATACCAGGTGCTTC |
| | | AAGAATTGCAT | CCCAAATATATAC | TCA |
| | | CA | | |
| PIV | D5 | CTGTGGTACAA | CAGCTTGTCCAAGTAG | AACTGGAAGAACATATT |
| | | AACAAAGCTAT | GAACATTTC | TGG |
| | | GCA | | |
| PIV | D6 | AAACTTAATCAC | CCGGGTTTAAATCAGG | AAACCTGATATGACTTC |
| | | TCAAGGATGTG | ATACATATCTGA | CC |
| | | CAGA | | |
| PIV | D7 | CTGACACAGCTT | AATCAATGTGCATATTC | CAGTTCGGAAAATGATT |
| | | GATCCACTCA | CAAGAATTGTGG | CTAC |
| PIV | D8 | CATTATGGAATG | GATGCTTTCTCTGCTCC | ACTGCATCCTTTGTTGAT |
| | | GAAACGGTGCA | TACTTTATGT | AAG |
| PIV | D9 | TGTAGACAATTT | TGGATCTCTCAGCTGTA | AAGTTAGCAATAATTGA |
| | | AGCACTGATCCT | ACTTAGCA | ATAAAC |
| | | AGGT | | |
| PIV | D10 | CATGAAGATTA | ATTGCAGAGGTGAATC | ATGCTTTTGGACCATTTT |
| | | CTTGTTCTGAAA | CACACA | C |
| | | ACAGTCC | | |
| PIV | D11 | CAGGGAAAGAT | TGCAACAAAGGAGCCC | TCTAGCAAGGAATTTAA |
| | | TCTTGGAGCTTA | ATGATAA | GAAAG |
| | | GA | | |
| PIV | D12 | CGAAGCAGCAA | GTGTGATTATGACTGG | CTTGGAGATCTAGATAA |
| | | CATTCCAGATGA | GCTGTTGA | TTAAAAC |
| | | TA | | |

In exemplary embodiments, the primer pair D includes at least two of the primer pairs as provided in Table 4, in a combination with the corresponding probe sequences in Table 4.

In exemplary embodiments, the method further includes a primer pair E in Table 5 and a probe for detection of endogenous internal control RNase P gene in Table 5:

TABLE 5

| | Primer | | | |
|---|---|---|---|---|
| virus | pair No. | forward primer | reverse primer | probe |
| RNaseP | E1 | AGATTTGGACCTGC | GAGCGGCTGTCTCCACAA | TTCTGACCTGAAGG |
| | | GAGCG | GT | CTCTGCGCG |
| RNaseP | E2 | GGACAGGCTTAAAG | GCTGCTTAGCATCTGTTT | AAGCAGAGAGAAA |
| | | CTATTGGACTT | TCTTCTC | TGC |
| RNaseP | E3 | AGCTTTTTCATATTG | CGCCACATTAATAGGAG | ATGCAGACTAAAT |
| | | CTTGCACACAT | GTCTTTTGA | CCACATCTA |
| RNaseP | E4 | ACCTTGGCTATTCA | TGGCAAAGTTGTGAAGA | CTGGTTTTTCAATT |
| | | GTTGTTGCTAT | GTTCAGA | TCC |
| RNaseP | E5 | CTGGAAAACAATCT | CGAGCAGCGCTTCTCCAT | ACAGTGAAGAAAC |
| | | TCATCTCCTTCTG | | CTCG |
| RNaseP | E6 | GAGCGGGTTCTGAC | AGCAACAACTGAATAGC | CTGTCTCCACAAGT |
| | | CTGAAG | CAAGGT | CCG |
| RNaseP | E7 | GCTTTTTTACTTTTG | TCGGCAGGCTTCTGTAAC | TAGGGTTTGGAATC |
| | | GGTGGTTTCCT | ATTAC | ACTTTC |
| RNaseP | E8 | GCTCCTCTGCTGGAT | GGAGACCGTGGAGAACA | CAAAGCGAAAACC |
| | | TGCA | GAAG | CCCGCCGG |
| RNaseP | E9 | CTCTGTTACAGTTAT | GTTGTTGCAGTTTTTCCA | CATCTAAATGTGTG |
| | | GCAGACTAAATCCA | AAGACAGAA | CAAGCAA |
| RNaseP | E10 | AAAACCAGTAGCTG | CAGTGAGATGGATCCGA | CACAACTTTGCCAA |
| | | TTTCTGAACTCTT | GACAATAA | TTGT |
| RNaseP | E11 | GCCGCTCACCTTGG | GAAGAGTTCAGAAACAG | CAGTTGTTGCTATC |
| | | CTATT | CTACTGGTT | AATCAT |
| RNaseP | E12 | CTTCTGATGGCCGA | TGCGGTGTCCACCAACTG | CCATGGAGAAACT |
| | | GGTTTCTT | | AGAAAAA |

In exemplary embodiments, the method includes at least one of the following primer pair and probe sets in Table 6 for the detection of adenovirus, metapneumovirus, rhinovirus/enterovirus, and/or parainfluenza in the sample:

TABLE 6

| | Primer | | | |
|---|---|---|---|---|
| virus | pair No. | forward primer | reverse primer | probe |
| Adeno | A2 | ACAAGGACCGCAT | CCACGAATCCTGAGTTGT | CCATGAGTAGG |
| | | GTATTCTTTCTT | TATGTTG | CAAGTTG |
| Adeno | A11 | CTAAAATACAAGC | GGAAGGACAAATGGATA | TTAGTGCAAGA |
| | | AAACCGCTGACT | CGCTGTAG | GGTTTTATG |
| Rhino/ | C1 | GGGTTGTTCCCAC | GGAATTAAAACAGGCGT | ACCAGAGTACT |
| Entero | | TCAAGGG | ACAAAGGT | AGCCGCCACG |
| Rhino/ | C2 | ATTGGTCTGGATC | CTGGAGGTGTGTAACAT | CTGTTGCCATAA |
| Entero | | TCTAGAAATGACA | AGGATCAA | AACTAC |
| | | TTC | | |
| MPV | B1 | TGAAATCAGAACA | AAAGTGTTGTGCAGCCTA | CAGTGTTTTAGT |
| | | AATTCTACCCAGG | GACA | TATTTAAAAAC |
| | | TT | | C |
| MPV | B4 | GCCTTATTATCTAT | CTTAGCTTGTCGTTCTGA | CAACACTAATG |
| | | AGAAAAGAATGA | TCCTACT | AGAGACCC |
| | | ACGTGCT | | |
| PIV | D5 | CTGTGGTACAAAA | CAGCTTGTCCAAGTAGG | AACTGGAAGAA |
| | | CAAAGCTATGCA | AACATTTC | CATATTTGG |
| PIV | D6 | AAACTTAATCACT | CCGGGTTTAAATCAGGAT | AAACCTGATAT |
| | | CAAGGATGTGCAG | ACATATCTGA | GACTTCCC |
| | | A | | |
| PIV | D7 | CTGACACAGCTTG | AATCAATGTGCATATTCC | CAGTTCGGAAA |
| | | ATCCACTCA | AAGAATTGTGG | ATGATTCTAC |
| PIV | D11 | CAGGGAAAGATTC | TGCAACAAAGGAGCCCA | TCTAGCAAGGA |
| | | TTGGAGCTTAGA | TGATAA | ATTTAAGAAAG |
| RNaseP | E3 | AGCTTTTTCATATT | CGCCACATTAATAGGAG | ATGCAGACTAA |
| | | GCTTGCACACAT | GTCTTTTGA | ATCCACATCTA |

In exemplary embodiments, the method includes at least one of the following primer pair and probe sets in Table 7 for the detection of adenovirus, metapneumovirus, rhinovirus/enterovirus, and/or parainfluenza in the sample:

TABLE 7

| | Primer | | | |
|---|---|---|---|---|
| virus | pair No. | forward primer | reverse primer | probe |
| Adeno | A3 | CCTCGATGAT | AACTTGTTTCCCAGGG | CCGGCGATGTGCAT |
| | | GCCGCA | TGAAG | GTAAGACCA |
| Adeno | A12 | ACCACCGTCA | GGCGTCAGTAACGGTC | CCTGCTCTCACAGA |
| | | GTGAAAACG | AC | TCACGGGAC |
| Rhino/ | C3 | GGTGTGAAG | GACACCCAAAGTAGTC | CTCCGGCCCCTGAA |
| Entero | | AGTCTATTGA | GGT | TG |
| | | GCT | | |
| Rhino/ | C4 | CTGCGTGGCG | CACGGACACCCAAAGT | CTCCGGCCCCTGAA |
| Entero | | GCC | AGTC | TG |
| Rhino/ | C5 | GCGTGGCTGC | ACACGGACACCCAAAG | CTCCGGCCCCTGAA |
| Entero | | CTGC | TAGT | TG |
| MPV | B5 | TTTGGGACAA | GCCAACTCCAGTAATT | TTGCCCCGCACTTCA |
| | | ATCATAATGT | GTGGTTA | TATTTGCA |
| | | CTCG | | |
| MPV | B6 | AGCTTTTAAG | CATCAATGTACCCTTG | CCTGCTCCTGATATT |
| | | AAACACAGA | AACCACAT | ATTGACAAACCA |
| | | TAAGGCT | | |
| PIV | D1 | GGAAGAAGC | TCTTCAAGCTCTGGAC | ATGACATCAGTTAT |
| | | ATGGAAACTA | TTGG | TCTTGTACTATGTGT |
| | | GCA | | GCTGCT |
| PIV | D2 | TGGACTTGGA | ACTTGGCCATGGGTCC | ATGCTGCAAGAGCC |
| | | ACAAGATGG | TA | AATGTAGGCC |
| | | C | | |
| PIV | D3 | CAAGACACA | GATTGAATTACACCAA | TCGATTGCTCTCTTC |
| | | AAAGACAAA | GATTCTGCA | TGTATCCTTCCT |
| | | TGGAAAGG | | |
| PIV | D4 | ACAGATGAA | TCATTATCACCAAAAG | CAAATACCAGGTGC |
| | | CGAAGAATTG | CCCAAATATATAC | TTCTCA |
| | | CATCA | | |
| RNaseP | E1 | AGATTTGGAC | GAGCGGCTGTCTCCAC | TTCTGACCTGAAGG |
| | | CTGCGAGCG | AAGT | CTCTGCGCG |

In exemplary embodiments, the method includes at least one of the following primer pair and probe sets in Table 8 for the detection of adenovirus, metapneumovirus, rhinovirus/enterovirus, and/or parainfluenza in the sample:

TABLE 8

| | Primer | | | |
|---|---|---|---|---|
| virus | pair No. | forward primer | reverse primer | probe |
| Adeno | A1 | ACCGTTTGGAT | GCCCTGATTTCCC | CCTGCATGTCCA |
| | | CCTCGAAAGC | GTTTCC | GATTT |
| Adeno | A5 | CTCGGAATGCC | GTCGATGTGACTG | CCTGTAGCTCAC |
| | | CACTACCA | AGAAGGTTTCAT | ATGGCT |
| Adeno | A6 | TCCCCATATGG | GCACAGATGAGT | ATGGTGAACAT |
| | | CTCTGAATGAA | ATGGTGAAGAAG | CAATCATG |
| | | ATC | A | |
| Rhino/ | C5 | GCGTGGCTGCC | ACACGGACACCC | CTCCGGCCCCTG |
| Entero | | TGC | AAAGTAGT | AATG |
| Rhino/ | C6 | CCTGGAGCCTT | AACACGGACACC | AAGGTCGTAAT |
| Entero | | GTGTCACA | CAAAGTAGTC | GAGCAATTC |
| Rhino/ | C7 | CGCATGTGCTT | GGCTCCAGGGTT | CTCCGGCCCCTG |
| Entero | | GGTTGTGATTC | AAGGTTAGC | AATG |
| MPV | B2 | TGGGTTAACTG | TTTATGCTTTTCA | TAGTTCCAAGA |
| | | AACAATTATCA | AGGTGTCCCCTAA | ACTCTAGATCTG |
| | | GTGTTGA | | |
| MPV | B3 | GAGAAGGCAA | CCCTGATTGTTGT | CAAGCGGTGAA |
| | | AAGAGCTCAA | CCTGAGTTTCTT | TCAGAAGA |
| | | CAAAA | | |
| PIV | D1 | GGAAGAAGCA | TCTTCAAGCTCTG | ATGACATCAGTT |
| | | TGGAAACTAG | GACTTGG | ATTCTTGTACTA |
| | | CA | | TGTGTGCTGCT |
| PIV | D9 | TGTAGACAATT | TGGATCTCTCAGC | AAGTTAGCAAT |
| | | TAGCACTGATC | TGTAACTTAGCA | AATTGAATAAA |
| | | CTAGGT | | C |
| PIV | D10 | CATGAAGATTA | ATTGCAGAGGTG | ATGCTTTTGGAC |
| | | CTTGTTCTGAA | AATCCACACA | CATTTTC |
| | | AACAGTCC | | |
| PIV | D11 | CAGGGAAAGA | TGCAACAAAGGA | TCTAGCAAGGA |
| | | TTCTTGGAGCT | GCCCATGATAA | ATTTAAGAAAG |
| | | TAGA | | |
| RNaseP | E2 | GGACAGGCTT | GCTGCTTAGCATC | AAGCAGAGAGA |
| | | AAAGCTATTGG | TGTTTTCTTCTC | AATGC |
| | | ACTT | | |

In exemplary embodiments, the method includes at least one of the following primer pair and probe sets in Table 9 for the detection of adenovirus, metapneumovirus, rhinovirus/enterovirus, and/or parainfluenza in the sample:

TABLE 9

| | Primer | | | |
|---|---|---|---|---|
| virus | pair No. | forward primer | reverse primer | probe |
| Adeno | 7A | GTACCTGACTTCCT | CCGATGCAGACAACATA | CTGGCGCTTGTAC |
| | | TTGACGAGAAA | GTAAAGC | CTC |
| Adeno | A8 | CATTGCTCGATCTC | CCTGAGGCGAAAAGAG | CAGTGTGGAAGAG |
| | | ACTGAAGTG | GTTCA | CTTAGCCGC |
| Rhino/ | C3 | GGTGTGAAGAGTC | GACACCCAAAGTAGTCG | CTCCGGCCCCTGA |
| Entero | | TATTGAGCT | GT | ATG |
| Rhino/ | C10 | ACAAATAGCATCC | CCACAAAACATTGTTAA | CCCACTCAAGAGA |
| Entero | | ATACATGACTCGA | ATAGCTGCTCT | AAAG |
| | | A | | |
| MPV | B7 | TCTCTTCAAGGGAT | TGTTGTTGTGCCTACAT | ACTGAGACTCTTT |
| | | TCACCTGAGT | CTCTCTTT | CAATATAGC |
| MPV | B8 | GCAATCAGCCATG | TCCAACAGCCTGAGGAT | CAGTGTAGCACGC |
| | | TGGATATTCTGT | CTCT | TCATT |
| PIV | D9 | TGTAGACAATTTAG | TGGATCTCTCAGCTGTA | AAGTTAGCAATAA |
| | | CACTGATCCTAGGT | ACTTAGCA | TTGAATAAAC |
| PIV | D10 | CATGAAGATTACTT | ATTGCAGAGGTGAATCC | ATGCTTTTGGACC |
| | | GTTCTGAAAACAG | ACACA | ATTTTC |
| | | TCC | | |
| PIV | D11 | CAGGGAAAGATTC | TGCAACAAAGGAGCCC | TCTAGCAAGGAAT |
| | | TTGGAGCTTAGA | ATGATAA | TTAAGAAAG |
| PIV | D12 | CGAAGCAGCAACA | GTGTGATTATGACTGGG | CTTGGAGATCTAG |
| | | TTCCAGATGATA | CTGTTGA | ATAATTAAAAC |
| RNaseP | E4 | ACCTTGGCTATTCA | TGGCAAAGTTGTGAAGA | CTGGTTTTTCAATT |
| | | GTTGTTGCTAT | GTTCAGA | TCC |

In exemplary embodiments, the method includes at least one of the following primer pair and probe sets in Table 10 for the detection of adenovirus, metapneumovirus, rhinovirus/enterovirus, and/or parainfluenza in the sample:

TABLE 10

| | Primer | | | |
|---|---|---|---|---|
| virus | pair No. | forward primer | reverse primer | probe |
| Adeno | A4 | CCTGCACCATTCCCAG | ACCTGTTTGTAATGT | TTCCAGCCTTG |
| | | ATAATTTTC | GTGGTTTGGA | AATTAT |
| Adeno | A10 | CTATCTATATAATATA | CACACAGCGCGCAC | CCAACATGTA |
| | | CCTTATAGATGGAATG | TTTT | AATGAGGTAA |
| | | GT | | TTT |
| Rhino/ | C11 | CTTGTGTGCATGTGAC | CATTGAACAACCCCT | CTGGCTTGGAT |
| Entero | | AGAAATCAA | CCTTCCT | AATCAC |
| Rhino/ | C12 | TCAGTTTGGTAGATAG | CACAGGCCCTTGAA | ATGATGGCAG |
| Entero | | GGTCTCTAATGTT | ATAACACTTC | TAACTAATTG |
| MPV | B10 | ACTGTGAAACAAGAG | CATTCTCTCGACTGC | CAGCAGCAGG |
| | | GTGATCATGT | TCAGCAA | GATCAA |
| MPV | B11 | GGGAAGCTAACAAGA | TCAGACTAACTTTAC | CCATGGCAGC |
| | | AATTACATGATACT | ATCCAGTAGAACTG | ATGTTAA |
| | | | A | |
| PIV | D4 | ACAGATGAACGAAGA | TCATTATCACCAAAA | CAAATACCAG |
| | | ATTGCATCA | GCCCAAATATATAC | GTGCTTCTCA |
| PIV | D5 | CTGTGGTACAAAACAA | CAGCTTGTCCAAGTA | AACTGGAAGA |
| | | AGCTATGCA | GGAACATTTC | ACATATTTGG |
| PIV | D6 | AAACTTAATCACTCAA | CCGGGTTTAAATCA | AAACCTGATA |
| | | GGATGTGCAGA | GGATACATATCTGA | TGACTTCCC |
| PIV | D7 | CTGACACAGCTTGATC | AATCAATGTGCATAT | CAGTTCGGAA |
| | | CACTCA | TCCAAGAATTGTGG | AATGATTCTAC |
| RNaseP | E4 | ACCTTGGCTATTCAGT | TGGCAAAGTTGTGA | CTGGTTTTTCA |
| | | TGTTGCTAT | AGAGTTCAGA | ATTTCC |

In exemplary embodiments, the method includes at least one of the following primer pair and probe sets in Table 11 for the detection of adenovirus, metapneumovirus, rhinovirus/enterovirus, and/or parainfluenza in the sample:

TABLE 11

| | Primer | | | |
|---|---|---|---|---|
| virus | pair No. | forward primer | reverse primer | probe |
| Adeno | A1 | ACCGTTTGGA | GCCCTGATTTCCCGTT | CCTGCATGTCCAGATTT |
| | | TCCTCGAAAG | TCC | |
| | | C | | |
| Adeno | A9 | ACTGGAGGCT | GCTCCGAGTAACGAAT | CTCCCTTCCGTAGGCGC |
| | | CCCTTCGTA | GCTGTT | |
| Rhino/ | C5 | GCGTGGCTGC | ACACGGACACCCAAA | CTCCGGCCCCTGAATG |
| Entero | | CTGC | GTAGT | |
| Rhino/ | C8 | CCTGGAGCCT | AACACGGACACCCAA | CCCGGAATTGCTCATTA |
| Entero | | TGTGTCACA | AGTAGTC | C |
| Rhino/ | C9 | GCCCCTGAAT | ACGACCTTATCATCAC | CCTTAACCCTGGAGCCT |
| Entero | | GCGGCTAA | TGGTTTGTG | T |
| MPV | B9 | TGTCTCTTCAA | TGCAGTTGTGGTGCCT | TCTGTCATATAAACATG |
| | | GGGATTCACC | ACATC | CTATATTAA |
| | | TAAGT | | |
| MPV | B12 | ACAGAGAAAG | TCTTGACTATTGTCTT | TCTTCTTCTTCAGATTCT |
| | | CAAAAGAGCT | GTATGTCTTTTGGTT | CC |
| | | TAACAAAATT | | |
| PIV | D1 | GGAAGAAGCA | TCTTCAAGCTCTGGAC | ATGACATCAGTTATTCT |
| | | TGGAAACTAG | TTGG | TGTACTATGTGTGCTGC |
| | | CA | | T |
| PIV | D6 | AAACTTAATC | CCGGGTTTAAATCAGG | AAACCTGATATGACTTC |
| | | ACTCAAGGAT | ATACATATCTGA | CC |
| | | GTGCAGA | | |
| PIV | D7 | CTGACACAGC | AATCAATGTGCATATT | CAGTTCGGAAAATGATT |
| | | TTGATCCACTC | CCAAGAATTGTGG | CTAC |
| | | A | | |
| PIV | D8 | CATTATGGAA | GATGCTTTCTCTGCTC | ACTGCATCCTTTGTTGA |
| | | TGGAAACGGT | CTACTTTATGT | TAAG |
| | | GCA | | |
| RNaseP | E8 | GCTCCTCTGCT | GGAGACCGTGGAGAA | CAAAGCGAAAACCCCC |
| | | GGATTGCA | CAGAAG | GCCGG |

### Samples

The disclosure provides kits and methods for the detection of respiratory pathogens in a sample. As will be appreciated by those in the art, the sample may include any number of components, including, but not limited to, nasal and/or throat washes, nasal and/or throat swabs, nasal aspirates obtained from human patients; research samples; purified samples, such as purified genomic DNA, RNA, proteins, etc.; and raw samples (e.g., bacteria, virus, genomic DNA, etc.). As will be appreciated by those in the art, any experimental manipulation could have been performed on the sample before analysis. In exemplary embodiments, the sample type for diagnosis of respiratory diseases is a human nasal or throat swab.

If present, the nucleic acid from the sample can be isolated using known techniques. For example, the sample can be treated with known lysis buffers, sonication, electroporation, etc., to lyse open cells, thereby releasing the nucleic acids in the cells. Purification of the nucleic acids resulting from lysis of the cells can occur if needed, as will be appreciated by those in the art. In addition, the reactions outlined herein can be accomplished in a variety of ways, as will be appreciated by those in the art. Components of the reaction can be added simultaneously, or sequentially, in any order, with the exemplary embodiments outlined below. In addition, the reaction can include a variety of other reagents that can be included in the assays. These reagents can include salts, buffers, neutral proteins, e.g. albumin, detergents, etc., which can be used to facilitate optimal hybridization and detection, and/or reduce non-specific or background interactions. Also reagents that otherwise improve the efficiency of the assay, such as protease inhibitors, nuclease inhibitors, anti-microbial agents, etc., can be used, depending on the sample preparation methods and purity of the target.

In exemplary embodiments, the sample includes nucleic acid extracted from a nasopharyngeal swab (NP) specimen. The specimen can be collected in Universal Transport Media (UTM) or Viral Transport Media (VTM) according to appropriate laboratory procedures. The nucleic acids can also be isolated and purified from the specimens using a nucleic acid isolation k, such as, e.g. the MagMAX™ Viral/Pathogen II Nucleic Acid Isolation Kit. Nucleic acid extraction can be performed via an automated process using, e.g., the KingFisher™ Flex Purification System. For RNA viruses, the RNA is reverse transcribed into cDNA. The cDNA and genomic DNA (from DNA viruses) are then subjected for amplification using the currently disclosed methods and kits.

### Reaction Mixture

The terms “reaction mixture,” “amplification mixture,” or “PCR mixture” as used herein refer to a mixture of components necessary to amplify at least one amplicon from nucleic acid templates. The mixture can include one or more nucleotides (dNTPs), a thermostable polymerase, primers as described herein, and a plurality of nucleic acid templates. The mixture can further include a buffer, a salt, and/or Mg²⁺.

The one or more nucleotides can be, for example, dTTP, dATP, dCTP, dGTP or dUTP. In an embodiment, the concentration of each of the nucleotides in the composition is about 0.5 mM to about 5 mM.

The thermostable polymerase can be a DNA polymerase. In an embodiment, the DNA polymerase is a thermostable DNA polymerase. For example, the thermostable DNA polymerase can be Taq DNA polymerase, a mutant, a variant, or a derivative thereof. In an embodiment, the concentration of the DNA polymerase(s) is between about 0.005 U/μL to about 0.5 U/μL.

The nucleic acid templates can be a ribonucleic acid template or a deoxyribonucleic acid template.

The buffer can include, for example, TRIS, TRICINE, BIS-TRICINE, HEPES, MOPS, TES, TAPS, PIPES, and/or CAPS.

The salt can be, for example, potassium chloride, potassium acetate, potassium sulfate, ammonium sulfate, ammonium chloride, ammonium acetate, magnesium chloride, magnesium acetate, magnesium sulfate manganese chloride, manganese acetate, manganese sulfate, sodium chloride, sodium acetate, lithium chloride and/or lithium acetate.

The working concentration range of each component is well known in the art and can be further optimized as needed by an ordinary skilled artisan.

### Amplification

“Amplification” as used herein denotes the use of any amplification procedures to increase the concentration of a particular nucleic acid sequence (e.g., an amplicon) within a mixture of nucleic acid sequences that is later detected.

Amplification involves the amplification (replication) of a sequence to be detected, such that the number of copies of the sequence is increased. Suitable amplification techniques include, but are not limited to, the polymerase chain reaction (PCR), strand displacement amplification (SDA), transcription mediated amplification (TMA) and nucleic acid sequence based amplification (NASBA).

In an exemplary embodiment, the amplification technique is PCR. The polymerase chain reaction (PCR) is widely used and described, and involves the use of primer extension combined with thermal cycling to amplify a target sequence. As used herein, “PCR”, unless specifically defined, refers to either single-plex or multiplex PCR assays, and can be real time or quantitative PCR (wherein detection occurs during amplification), end-point PCR (when detection occurs at the end amplification), or reverse transcription PCR, including but not limited to, “real-time PCR” or “quantitative PCR” or “qPCR”, “digital PCR” or “dPCR”, “reverse transcriptase PCR” or “RT-PCR”, “multiplex PCR”, “nested PCR”, “hot start PCR”, “long-range PCR”, “assembly PCR”, “asymmetric PCR”, “in situ PCR,” “single-cell PCR,” or “fast-cycling PCR,” among others.

Exemplary embodiments of amplification are not limited to PCR. For instance, signal amplification, single base extension (SBE) or minisequencing, oligonucleotide ligation amplification (OLA) and/or rolling-circle amplification can be used for amplification. In an exemplary embodiment, amplification includes OLA followed by RCA.

A skilled person would be well aware of the real time PCR systems that can be used for a multiplex detection using several dyes. Exemplary systems include Real-time PCR (qPCR) instrument, including for example a QuantStudio Real-Time PCR system, such as the QuantStudio™ 5 RealTime PCR System (QS5), QuantStudio™ 7 RealTime PCR System (QS7), and/or QuantStudio™ 12K Flex System (QS12K), or a 7500 Real-Time PCR system, such as the 7500 Fast Dx system, from Thermo Fisher Scientific.

In exemplary embodiments, the nucleic acid amplification assays as described herein are performed using a Real-time PCR (qPCR) instrument, including for example a QuantStudio™ Real-Time PCR system, such as the QuantStudio™ 5 RealTime PCR System (QS5) and QuantStudio™ 12K Flex System (QS12K), or a 7500 Real-Time PCR system, such as the 7500 Fast Dx system, from Thermo Fisher Scientific.

### Amplicon

The terms “amplified product” or “amplicon” refer to a fragment of DNA amplified by a polymerase using a pair of primers in an amplification method, such as PCR.

“Probe” as used herein, is a non-extendable oligonucleotide attached to a fluorescent reporter dye and a quencher moiety.

“Primer” as used herein can refer to more than one primer and refers to an oligonucleotide, whether occurring naturally or produced synthetically, which is capable of acting as a point of initiation of synthesis when placed under conditions in which synthesis of a primer extension product, which is complementary to a nucleic acid strand, is induced in the presence of nucleotides and an agent for polymerization, such as DNA polymerase, at a suitable temperature for a sufficient amount of time and in the presence of a buffering agent. Such conditions can include, for example, the presence of at least four different deoxyribonucleoside triphosphates (such as G, C, A, and T) and a polymerization-inducing agent such as DNA polymerase or reverse transcriptase, in a suitable buffer (“buffer” includes substituents which are cofactors, or which affect pH, ionic strength, etc.), and at a suitable temperature. In exemplary embodiments, the primer can be single-stranded for maximum efficiency in amplification. The primers herein are selected to be substantially complementary to the different strands of each specific sequence to be amplified. This means that the primers must be sufficiently complementary to hybridize with their respective strands. A non-complementary nucleotide fragment can be attached to the 5′-end of the primer, with the remainder of the primer sequence being complementary, or partially complementary, to the target region of the target nucleic acid. Commonly, the primers are complementary, except when non-complementary nucleotides may be present at a predetermined sequence location, such as a primer terminus as described.

The complement of a nucleic acid sequence as used herein refers to an oligonucleotide which, when aligned with the nucleic acid sequence, the 5′ end of one sequence is paired with the 3′ end of the other, is in “antiparallel association.” Complementarity need not be perfect; stable duplexes can contain mismatched base pairs or unmatched bases.

The primers and/or probes described herein can further include a fluorescent or other detectable label. In exemplary embodiments the primers and/or probes may further include a quencher and, in other exemplary embodiments, the probes can further include a minor groove binder (MGB) moiety. Suitable fluorescent labels include, but are not limited to, Alexa Fluor™ dyes (e.g., AF647 and/or AF676), ABY™, VIC™, JUN™, FAM™, HEX™, NED™ and Cy5 dyes. For 2-plex reactions, the most popular combination is FAM™ and HEX™ (JOE/VIC™); for 3-plex reactions, FAM™, HEX™ (JOE/VIC™), and Cy5 or NED™, FAM™, and VIC™; and for 4-plex or 5-plex, FAM™, HEX™ (JOE/VIC™), Texas Red™, and Cy5 dyes, or FAM™, VIC™, ABY™, and JUN™. Suitable quenchers include but are not limited to QSY™ (e.g., QSY7 and QSY21), BHQ™ (Black Hole Quencher) and DFQ (Dark Fluorescent Quencher), and Dabsyl and Dabcel sulfonate/carboxylate Quenchers. Detector probes can also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes can also include sulfonate derivatives of fluorescein dyes with SO₃ instead of the carboxylate group, phosphoramidite forms of fluorescein, and/or phosphoramidite forms of Cy5.

It should be appreciated that when using more than one detectable label, particularly in a multiplex format, each detectable label preferably differs in its spectral properties from the other detectable labels used therewith such that the labels can be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels can include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple™, VIC™, ABY™, JUN™; FITC; 6-carboxy-4′,5′-dichloro-2,7″-dimethoxy-fluorescein (JOE)); 6-carboxy-1,4-dichloro-2′,7′-dichloro-fluorescein (TET); 6-carboxy-1,4-dichloro-2′,4′,5′,7′-tetra-chlorofluorescein (HEX); Alexa Fluor™ fluorophores (e.g., 350, 405, 430, 488, 500, 514, 532, 546, 555, 568, 594, 610, 633, 635, 647, 660, 680, 700, 750); BODIPY™ fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue®, Cascade Yellow: Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, IAEDANS/fluorescein, EDANS/dabcyl, BODIPY™ FL/BODIPY™ FL, Fluorescein/QSY7 and QSY9), LysoTracker™ and LysoSensor™ (e.g., LysoTracker™ Blue DND-22, LysoTracker™ Blue-White DPX, LysoTracker™ Yellow HCK-123, LysoTracker™ Green DND-26, LysoTracker™ Red DND-99, LysoSensor™ Blue DND-167, LysoSensor™ Green DND-189, LysoSensor™ Green DND-153, LysoSensor™ Yellow/Blue DND-160, LysoSensor™ Yellow/Blue 10,000 MW dextran), Oregon Green™ (e.g., 488, 488-X, 500, 514): rhodamines (e.g., 110, 123, B, B 200, BB, BG, B extra, 5-carboxy tetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl-rhodamine), Tetramethylrhodamine (TRITC), WT), Texas Red®, Texas Red®-X, among others as would be known to those of skill in the art.

Other detectable labels can be used in addition to or as an alternative to labelled probes. For example, primers can be labeled and used to both generate amplicons and to detect the presence (or concentration) of amplicons generated in the reaction, and such can be used in addition to or as an alternative to labeled probes described herein. As an example, primers can be labeled and utilized as described in Nazarenko et al. (Nucleic Acids Res. 2002 May 1; 30 (9): e37), Hayashi et al. (Nucleic Acids Res. 1989 May 11; 17 (9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, can be used to detect amplified target nucleic acids. In exemplary embodiments, intercalating labels can be used such as ethidium bromide, SYBR® Green I, SYBR® GreenER, and PicoGreen® (Life Technologies Corp., Carlsbad, CA), thereby allowing visualization in real-time, or end point, of an amplification product in the absence of a detector probe. In exemplary embodiments, real-time visualization can include both an intercalating detector probe and a sequence-based detector probe. In exemplary embodiments, the detector probe is at least partially quenched when not hybridized to a complementary sequence in the amplification reaction and is at least partially unquenched when hybridized to a complementary sequence in the amplification reaction. In some embodiments, probes can further include various modifications such as a minor groove binder to further provide desirable thermodynamic characteristics.

In exemplary embodiments, the amplicon is labeled by incorporation of or hybridization to a labeled primer. In exemplary embodiments, the amplicon is labeled by hybridization to a labeled probe. In exemplary embodiments, the amplicon is labeled by binding of a DNA-binding dye. In exemplary embodiments, the dye is a single-strand DNA binding dye. In exemplary embodiments, the dye is a double-stranded DNA binding dye. In exemplary embodiments, the amplicon is labeled via polymerization or incorporation of labeled nucleotides in a template-dependent (or template-independent) polymerization reaction. This can be part of the amplifying step or alternatively the labeled nucleotide can be added after amplifying is completed. The labeled amplicon (or labeled derivative thereof) can be detected using any suitable method such as, for example, electrophoresis, hybridization-based detection (e.g., microarray, molecular beacons, and the like), chromatography, NMR, and the like.

In exemplary embodiments, the labeled amplicon is detected using capillary electrophoresis. In exemplary embodiments, the labeled amplicon is detected using qPCR. In exemplary embodiments, a plurality of different amplicons is formed, and optionally labeled, within a single reaction volume via a single amplification reaction. For example, a multiplex reaction (e.g., 2-plex, 3-plex, 4-plex, 5-plex, 6-plex) carried out in a single tube or reaction vessel (e.g., “single-tube” or “1-tube” or “single-vessel” reaction) can produce a plurality of amplicons that are labeled. In exemplary embodiments, the plurality of amplicons can be differentially labeled. In exemplary embodiments, each of the plurality of amplicons produced during amplification is labeled with a different label.

Optionally, in exemplary embodiments, a control template and/or assay, such as bacteriophage MS2 or RNase P control, is included in the kit. If the positive control sequence is an endogenously-derived control, such as RNase P, the presence of patient-derived nucleic acid (e.g., genomic DNA coding for RNase P, RNase P RNA, and/or reverse transcribed RNase P transcript), can be used as the template for an RNase P qPCR assay.

Specific combinations of virus detection probes and dyes used:

In exemplary embodiments, the method includes performing a 4-plex amplification assay with probes specific for amplicons generated using at least one primer pair A (target-adenovirus) labeled with Alexa Fluor™; with the probes specific for amplicons generated using at least one primer pair B (target-metapneumovirus) labeled with ABY™; with the probes specific for amplicons generated using at least one primer pair C (target-rhinovirus/enterovirus) labeled with VIC™; with probes specific for amplicons generated using at least one primer pair D (target-parainfluenza) labeled with FAM™. In exemplary embodiments, the method includes performing a 5-plex amplification assay with the probes mentioned above and, additionally, with probes specific for amplicons generated using at least one primer pair E (target-internal control RNase P gene) labeled with JUN™.

Another aspect provided herein is a method of detecting or quantifying a target nucleic acid molecule in a sample by polymerase chain reaction (PCR), such as by quantitative real-time polymerase chain reaction (qPCR). In exemplary embodiments, the method includes: (i) contacting a sample including one or more target nucleic acid molecules with a) at least one probe, such as those described herein, being sequence specific for the target nucleic acid molecule, where the at least one probe undergoes a detectable change in fluorescence upon amplification of the one or more target nucleic acid molecules; and with b) at least one oligonucleotide primer pair; (ii) incubating the mixture of step (i) with a DNA polymerase under conditions sufficient to amplify one or more target nucleic acid molecules; and (iii) detecting the presence or absence, or quantifying the amount, of the amplified target nucleic acid molecules by measuring fluorescence of the probe. In exemplary embodiments, the DNA polymerase includes 5′ exonuclease activity. In exemplary embodiments, the DNA polymerase is a Thermus aquaticus (Taq) DNA polymerase. In exemplary embodiments, the probe is a hydrolysis probe, such as a TaqMan® probe.

Another aspect provided herein is a kit for PCR, such as quantitative real-time polymerase chain reaction (qPCR) and reverse transcription polymerase chain reaction (RT-PCR). In exemplary embodiments the kit includes a probe, such as those described herein, instructions for conducting the PCR, and one or more of the following: a buffering agent, deoxynucleotide triphosphates (dNTPs), an organic solvent, an enzyme, enzyme cofactors, and an enzyme inhibitor. In exemplary embodiments, the kit for PCR includes a dye and/or quencher moiety described herein, instructions for conjugating or labeling the dye and/or quencher moiety to a biomolecule, such as an oligonucleotide, instructions for conducting the PCR, and one or more of the following: a buffering agent, deoxynucleotide triphosphates (dNTPs), an organic solvent, an enzyme, enzyme cofactors, and an enzyme inhibitor.

In yet further aspects provided herein are compositions, such as a “master mix”, for PCR including at least one probe as described herein, along with other components that are used in PCR. In exemplary embodiments, the composition is prepared such that it requires less than a 3× dilution prior to use in PCR, e.g., 2× dilution, 1.5× dilution, 1.2× dilution, etc.

In yet further aspects provided herein are systems for performing the methods described herein and/or for using the compositions and/or kits described herein. In exemplary embodiments, the system is a “point-of-service” (POS) system.

In exemplary embodiments, the system is a POS system located at a “point-of-care” (POC) location. In exemplary embodiments, the POS system is configured to collect and/or analyze samples from a subject at the POC location and does not require specialized equipment. In exemplary embodiments, the POS system is configured to produce rapid and easy-to-read visual results. In exemplary embodiments, the POS system is configured to perform an analysis in the field, in a home setting, and/or by a lay person not having specialized skills. In exemplary embodiments, POS system is configured to analyze a small-volume clinical sample in a short period of time (e.g., within hours or minutes).

In exemplary embodiments, the POS system is utilized at a location that is capable of providing a service (e.g., testing, monitoring, treatment, diagnosis, guidance, sample collection, verification of identity (ID verification), and other services) at or near the site or location of a subject. A service can be a medical service or it can be a non-medical service. In exemplary embodiments, the POS system provides a service at a predetermined location, such as a subject's home, school, or work, or at a grocery store, a drug store, a community center, a clinic, a doctor's office, a hospital, an outdoor triage tent, a makeshift hospital, a border check point, etc. The POS system can include one or more point of service devices, such as a portable virus/pathogen detector. In exemplary embodiments, the POS system is a point of care system. In exemplary embodiments, the POS system is suitable for use by non-specialized workers or personnel, such as nurses, police officers, civilian volunteers, or the patient.

In exemplary embodiments, the system is configured utilize a POC system at a location at which medical-related care (e.g., treatment, testing, monitoring, diagnosis, counseling, etc.) is provided. The POC system can be, e.g., at a subject's home, work, or school, or at a grocery store, a community center, a drug store, a doctor's office, a clinic, a hospital, an outdoor triage tent, a makeshift hospital, a border check point, etc. The POC system can be a system which aids in, or provides medical-related care, and can be located at or near the site or location of the subject or the subject's health care provider (e.g., subject's home, work, or school, or at a grocery store, a community center, a drug store, a doctor's office, a clinic, a hospital, etc.).

In exemplary embodiments, the system is configured to accept a clinical sample obtained from a subject at an associated POS location. In exemplary embodiments, the system is configured to analyze the clinical sample at the POS location. In exemplary embodiments, the system is configured to analyze a small volume clinical sample. In exemplary embodiments, the system is configured to analyze the clinical sample in a short period of time (within hours or minutes). In exemplary embodiments, the short period of time is determined with respect to the time at which sample analysis began. In exemplary embodiments, the short period of time is determined with respect to the time at which the sample was inserted into a device for the analysis of the sample. In exemplary embodiments, the short period of time is determined with respect to the time at which the sample was obtained from the subject.

In exemplary embodiments, the system is configured to perform any of the amplification-based methods described herein and/or use any of the compositions and/or kits disclosed herein, including any of the described assays and/or assay panels. Such assays are contemplated for use with both thermal cycling amplification workflows and protocols, such as in PCR, as well as isothermal amplification workflows and protocols, such as in LAMP.

In exemplary embodiments, the system is configured to self-collect a biological sample, such as a nasal swab or a saliva sample. In exemplary embodiments, the self-collection can include the use of a self-collection kit and/or device, such as a swab or a tube (e.g., a saliva collection tube or similar saliva collection device). In exemplary embodiments, the self-collection kit includes instructions for use, including collection instructions, sample preparation or storage instructions, and/or shipping instructions. For example, the self-collection kit and/or device can be used by an individual, such as lay person, not having specialized skills or medical expertise. In exemplary embodiments, self-collection can be performed by the subject themselves or by any other individual in proximity to the subject, such as but not limited to a parent, a care giver, a teacher, a friend, or other family member.

Notably, in exemplary embodiments, the nucleic acid amplification protocol can be configured for rapid processing (e.g., in less than about 45 minutes) and high throughput, allowing for a minimally invasive method to quickly screen large numbers of individuals in a scalable way. This can be particularly useful to perform asymptomatic testing (e.g., high frequency/widespread testing at schools, workplaces, conventions, sporting events, large social gatherings, etc.) or for epidemiological purposes. The disclosed embodiments can also beneficially provide a lower cost sample collection system and methods that enable self-collection (reducing health care professional staffing needs) using a low-cost collection device. This eliminates the requirements for swabs, buffers, virus transmission media (or other specialized transport medium), and the like. The disclosed embodiments also allow for a reduction in Personal Protective Equipment (PPE) requirements and costs. Because the reagents and methods are streamlined (e.g., no precursor nucleic acid purification and/or extraction step), there is a reduced use of nucleic acid preparation plastics which brings a coincident reduction in reagent costs and inventory costs. There is also a beneficial reduced dependence on supply-constrained items, and the compatibility of these methods and kit components with existing equipment improves the flexibility and simplicity of their implementation to the masses. Overall, such embodiments allow for a less expensive assay that can be accomplished more quickly from sample collection through result generation.

The present disclosure includes, but is not limited to, embodiments represented by the following clauses.

Clause 1: An in vitro method for determining the presence or absence of adenovirus, metapneumovirus, rhinovirus/enterovirus, and parainfluenza in a sample, said method comprising the steps of: providing a reaction mixture that includes the sample and at least one primer pair set: wherein the at least one primer pair set includes at least one primer pair A that specifically amplifies a portion of adenovirus genome: at least one primer pair B that specifically amplifies a portion of metapneumovirus genome: at least one primer pair C that specifically amplifies a portion of rhinovirus/enterovirus genome; and at least one primer pair D that specifically amplifies a portion of parainfluenza genome; and (b) subjecting the reaction mixture to reaction conditions suitable to amplify targeted nucleic acids, thereby generating at least one amplicon: wherein the presence or absence of the at least one amplicon in the sample indicates the presence or absence of adenovirus, metapneumovirus, rhinovirus/enterovirus, and/or parainfluenza in the sample.

Clause 2: The method of clause 1, wherein the generating of the at least one amplicon includes subjecting the reaction mixture to PCR conditions.

Clause 3: The method according to clause 1, wherein: the amplicon produced using the at least one primer pair A comprises a probe sequence selected from the sequences specific for adenovirus in FIG. 1: the amplicon produced using the at least one primer pair B comprises a probe sequence selected from the sequences specific for metapneumovirus in FIG. 1, the amplicon produced using the at least one primer pair C comprises a probe sequence selected from the sequences specific for rhinovirus/enterovirus in FIG. 1, and/or the amplicon produced using the at least one primer pair D comprises a probe sequence selected from the sequences specific for parainfluenza in FIG. 1.

Clause 4: The method according to any one of the preceding clauses, wherein the reaction mixture contains probes specific for the amplicons generated in step b.

Clause 5: The method according to clause 1, wherein the reaction mixture comprises probes that are specific for amplicons generated using a specific forward and reverse primer pair, wherein the probes are selected from the probe sequences provided in FIG. 1.

Clause 6: The method according to any one of the preceding clauses, wherein: primer pair A comprises at least one forward and reverse primer pair sequence in FIG. 1 specific for adenovirus, a primer pair B comprises at least one forward and reverse primer pair sequence in FIG. 1 specific for metapneumovirus, a primer pair C comprises at least one forward and reverse primer pair sequence in FIG. 1 specific for rhinovirus/enterovirus, and a primer pair D comprises at least one forward and reverse primer pair sequence in FIG. 1 specific for parainfluenza.

Clause 7: The method according to clause 6, wherein the reaction mixture further comprises probes that detect amplicons produced from primer pair A, primer pair B, primer pair C and primer pair D, said probes having a sequence in FIG. 1 specific for detecting the amplicons.

Clause 8: The method according to any one of the preceding clauses, wherein the reaction mixture comprises an additional primer pair set, the additional primer pair set including: an additional primer pair A comprising at least one forward and reverse primer pair sequence in Table 1; an additional primer pair B comprising at least one forward and reverse primer pair sequence in Table 2; an additional primer pair C comprising at least one forward and reverse primer pair sequence in Table 3; and/or an additional primer pair D comprising at least one forward and reverse primer pair sequence in Table 4.

Clause 9: The method according to any one of the preceding clauses, wherein the reaction mixture further contains a primer pair E and, optionally, a probe, that specifically amplify at least a portion of a human RNase P gene.

Clause 10: The method of clause 9, wherein the primer pair E comprises at least one forward and reverse primer pair sequence in Table 5 and, when present, the probe that specifically amplifies at least a portion of the human RNase P gene comprises at least one probe sequence in Table 5.

Clause 11: The method according to any one of the preceding clauses, wherein the probes each contain a fluorescent reporter.

Clause 12: The method of clause 11, wherein the probes each contain a quencher.

Clause 13: The method of clause 11, wherein each of the probes is labeled at or near the 5′ end with a dye selected from Alexa Fluor™, ABY™, VIC™, JUN™, and FAM™.

Clause 14: The method of clauses 11 or 13, wherein each of the probes is labeled at the 3′ end with a quencher selected from QSY™, MGBNFQ, BHQ™, and DFQ.

Clause 15: The method of clause 13, wherein: probes specific for amplicons generated using the at least one primer pair A and/or the additional primer pair A are labeled with Alexa Fluor™, probes specific for amplicons generated using the at least one primer pair B and/or the additional primer pair B are labeled with ABY™, probes specific for amplicons generated using the at least one primer pair C and/are labeled with VIC™, probes specific for amplicons generated using the at least one primer pair D and/or the additional primer pair D are labeled with FAM™.

Clause 16: The method of clause 11, wherein the probe that specifically amplifies the at least a portion of the human RNase P gene is labeled with JUN™.

Clause 17: A composition for determining the presence or absence of adenovirus, metapneumovirus, rhinovirus/enterovirus, and/or parainfluenza in a sample, the composition including at least one primer pair set, wherein the at least one primer pair set includes at least one primer pair A that specifically amplifies a portion of adenovirus genome; at least one primer pair B that specifically amplifies a portion of metapneumovirus genome; at least one primer pair C that specifically amplifies a portion of rhinovirus/enterovirus genome; and at least one primer pair D that specifically amplifies a portion of parainfluenza genome.

Clause 18: The composition of clause 17, wherein the at least one primer pair A comprises at least one forward and reverse primer pair sequence in FIG. 1 specific for adenovirus, the at least one primer pair B comprises at least one forward and reverse primer pair sequence in FIG. 1 specific for metapneumovirus, the at least one primer pair C comprises at least one forward and reverse primer pair sequence in FIG. 1 specific for rhinovirus/enterovirus, and the at least one primer pair D comprises at least one forward and reverse primer pair sequence in FIG. 1 specific for parainfluenza.

Clause 19: The composition according to any one of the preceding clauses, further comprising probes specific for the amplicons produced using the at least one primer pair A, the at least one primer pair B, the at least one primer pair C, and the at least one primer pair D.

Clause 20: The composition according to clause 19, wherein the probes comprise a probe sequence provided in FIG. 1 that is specific for the amplicons produced using the at least one primer pair A, the at least one primer pair B, the at least one primer pair C, and the at least one primer pair D.

Clause 21: The composition according to any one of the preceding clauses, further comprising a polymerase, a buffer, and nucleotides.

Clause 22: The composition according to any one of the preceding clauses, further comprising a sample.

Clause 23: The composition according to any one of the preceding clauses, wherein the composition further comprises a primer pair E and, optionally, at least one probe, that specifically amplify at least a portion of a human RNase P gene.

Clause 24: The composition of clause 23, wherein the primer pair E comprises at least one forward and reverse primer pair sequence in Table 5 and, when present, the at least one probe that specifically amplifies at least a portion of the human RNase P gene comprises at least one probe sequence in Table 8.

Clause 25: The composition according to any one of clauses 19-24, wherein the probes contain a fluorescent reporter.

Clause 26: The composition according to any one of clauses 19-25, wherein the probes contain a quencher.

Clause 27: The composition according to any one of clauses 19-26, wherein the probes are labeled at or near the 5′ end with a dye selected from Alexa Fluor™, ABY™, VIC™, JUN™, and FAM™.

Clause 28: The composition according to any one of clauses 19-27, wherein the probes are labeled at the 3′ end with a quencher selected from QSY™, MGBNFQ, BHQ™, and DFQ.

Clause 29: The composition according to any one of clauses 19-28, wherein: probes specific for amplicons generated using the at least one primer pair A are labeled with Alexa Fluor™, probes specific for amplicons generated using the at least one primer pair B are labeled with ABY™, probes specific for amplicons generated using the at least one primer pair C are labeled with VIC™, and probes specific for amplicons generated using the at least one primer pair D are labeled with FAM™.

Clause 30: The composition according to any one of clauses 19-29, wherein the probe specific for amplifying at least a portion of the human RNase P gene is labeled with JUN™.

Clause 31: A kit for determining the presence or absence of adenovirus, metapneumovirus, rhinovirus/enterovirus, and/or parainfluenza in a sample, comprising a composition of any of clauses 17-30.

Clause 32: The kit according to clause 31, further comprising a primer pair E and, optionally, a probe that specifically amplify at least a portion of a human RNase P gene.

Clause 33: The kit according to clause 32, wherein the primer pair E comprises at least one forward and reverse primer pair sequence in Table 5 and, when present, the probe that specifically amplifies at least a portion of the human RNase P gene sequence comprises at least one probe sequence in Table 5.

Clause 34: The method, composition or kit according to any preceding clauses, wherein the at least one primer pair set and/or the additional primer pair set comprise at least one primer pair having a forward and reverse primer pair sequence as provided in Table 6, Table 7, Table 8, Table 9, Table 10 or Table 11; and wherein at least one of the probes has a probe sequence provided in Table 6, Table 7, Table 8, Table 9, Table 10 or Table 11.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatuses disclosed herein may be made without departing from the scope of the disclosure or of the invention. Thus, while various aspects and embodiments have been disclosed herein, other aspects and embodiments are contemplated. All changes that come within the meaning and range of equivalency of the claims are to be embraced within their scope.

1. An in vitro method for determining the presence or absence of adenovirus, metapneumovirus, rhinovirus/enterovirus, and parainfluenza in a sample, said method comprising the steps of:
(a) providing a reaction mixture that includes the sample and at least one primer pair set;
wherein the at least one primer pair set includes at least one primer pair A that specifically amplifies a portion of adenovirus genome;
at least one primer pair B that specifically amplifies a portion of metapneumovirus genome;
at least one primer pair C that specifically amplifies a portion of rhinovirus/enterovirus genome; and
at least one primer pair D that specifically amplifies a portion of parainfluenza genome; and
(b) subjecting the reaction mixture to reaction conditions suitable to amplify targeted nucleic acids, thereby generating at least one amplicon;
wherein the presence or absence of the at least one amplicon in the sample indicates the presence or absence of adenovirus, metapneumovirus, rhinovirus/enterovirus, and/or parainfluenza in the sample.

2. The method of claim 1, wherein the generating of the at least one amplicon includes subjecting the reaction mixture to PCR conditions.

3. The method according to claim 1, wherein:
the amplicon produced using the at least one primer pair A comprises a probe sequence selected from the sequences specific for adenovirus in FIG. 1;
the amplicon produced using the at least one primer pair B comprises a probe sequence selected from the sequences specific for metapneumovirus in FIG. 1,
the amplicon produced using the at least one primer pair C comprises a probe sequence selected from the sequences specific for rhinovirus/enterovirus in FIG. 1, and/or
the amplicon produced using the at least one primer pair D comprises a probe sequence selected from the sequences specific for parainfluenza in FIG. 1.

4. The method according to claim 1, wherein the reaction mixture contains probes specific for the amplicons generated in step b.

5. The method according to claim 1, wherein the reaction mixture comprises probes that are specific for amplicons generated using a specific forward and reverse primer pair, wherein the probes are selected from the probe sequences provided in FIG. 1.

6. The method according to claim 1, wherein:
primer pair A comprises at least one forward and reverse primer pair sequence in FIG. 1 specific for adenovirus,
a primer pair B comprises at least one forward and reverse primer pair sequence in FIG. 1 specific for metapneumovirus,
a primer pair C comprises at least one forward and reverse primer pair sequence in FIG. 1 specific for rhinovirus/enterovirus, and
a primer pair D comprises at least one forward and reverse primer pair sequence in FIG. 1 specific for parainfluenza.

7. The method according to claim 6, wherein the reaction mixture further comprises probes that detect amplicons produced from primer pair A, primer pair B, primer pair C and primer pair D, said probes having a sequence in FIG. 1 specific for detecting the amplicons.

8. The method according to claim 1, wherein the reaction mixture comprises an additional primer pair set, the additional primer pair set including:
an additional primer pair A comprising at least one forward and reverse primer pair sequence in Table 1;
an additional primer pair B comprising at least one forward and reverse primer pair sequence in Table 2;
an additional primer pair C comprising at least one forward and reverse primer pair sequence in Table 3; and/or
an additional primer pair D comprising at least one forward and reverse primer pair sequence in Table 4.

9. The method according to claim 1, wherein the reaction mixture further contains a primer pair E and, optionally, a probe, that specifically amplify at least a portion of a human RNase P gene.

10. The method of claim 9, wherein the primer pair E comprises at least one forward and reverse primer pair sequence in Table 5 and, when present, the probe that specifically amplifies at least a portion of the human RNase P gene comprises at least one probe sequence in Table 5.

11. The method according to claim 4, wherein the probes each contain a fluorescent reporter.

12. The method of claim 11, wherein the probes each contain a quencher.

13. The method of claim 11, wherein each of the probes is labeled at or near the 5′ end with a dye selected from Alexa Fluor™, ABY™, VIC™, JUN™, and FAM™.

14. The method of claim 11, wherein each of the probes is labeled at the 3′ end with a quencher selected from QSY™, MGBNFQ, BHQ™, and DFQ.

15. The method of claim 13, wherein:
probes specific for amplicons generated using the at least one primer pair A and/or the additional primer pair A are labeled with Alexa Fluor™,
probes specific for amplicons generated using the at least one primer pair B and/or the additional primer pair B are labeled with ABY™,
probes specific for amplicons generated using the at least one primer pair C and/are labeled with VIC™,
probes specific for amplicons generated using the at least one primer pair D and/or the additional primer pair D are labeled with FAM™.

16. The method of claim 11, wherein:
the probe that specifically amplifies the at least a portion of the human RNase P gene is labeled with JUN™.

17. A composition for determining the presence or absence of adenovirus, metapneumovirus, rhinovirus/enterovirus, and/or parainfluenza in a sample, the composition comprising:
at least one primer pair set, wherein the at least one primer pair set includes
at least one primer pair A that specifically amplifies a portion of adenovirus genome;
at least one primer pair B that specifically amplifies a portion of metapneumovirus genome;
at least one primer pair C that specifically amplifies a portion of rhinovirus/enterovirus genome; and
at least one primer pair D that specifically amplifies a portion of parainfluenza genome.

18. The composition of claim 17, wherein:
the at least one primer pair A comprises at least one forward and reverse primer pair sequence in FIG. 1 specific for adenovirus,
the at least one primer pair B comprises at least one forward and reverse primer pair sequence in FIG. 1 specific for metapneumovirus,
the at least one primer pair C comprises at least one forward and reverse primer pair sequence in FIG. 1 specific for rhinovirus/enterovirus, and
the at least one primer pair D comprises at least one forward and reverse primer pair sequence in FIG. 1 specific for parainfluenza.

19. The composition according to claim 17, further comprising probes specific for the amplicons produced using the at least one primer pair A, the at least one primer pair B, the at least one primer pair C, and the at least one primer pair D.

20. The composition according to claim 19, wherein the probes comprise a probe sequence provided in FIG. 1 that is specific for the amplicons produced using the at least one primer pair A, the at least one primer pair B, the at least one primer pair C, and the at least one primer pair D.

21. The composition according to claim 17, further comprising a polymerase, a buffer, and nucleotides.

22. The composition according to claim 17, further comprising a sample.

23. The composition according to claim 17, wherein the composition further comprises a primer pair E and, optionally, at least one probe, that specifically amplify at least a portion of a human RNase P gene.

24. The composition of claim 23, wherein the primer pair E comprises at least one forward and reverse primer pair sequence in Table 5 and, when present, the at least one probe that specifically amplifies at least a portion of the human RNase P gene comprises at least one probe sequence in Table 8.

25. The composition according to claim 19, wherein the probes contain a fluorescent reporter.

26. The composition according to claim 19, wherein the probes contain a quencher.

27. The composition according to claim 19, wherein the probes are labeled at or near the 5′ end with a dye selected from Alexa Fluor™, ABY™, VIC™, JUN™, and FAM™.

28. The composition according to claim 19, wherein the probes are labeled at the 3′ end with a quencher selected from QSY™, MGBNFQ, BHQ™, and DFQ.

29. The composition according to claim 19, wherein:
probes specific for amplicons generated using the at least one primer pair A are labeled with Alexa Fluor™,
probes specific for amplicons generated using the at least one primer pair B are labeled with ABY™,
probes specific for amplicons generated using the at least one primer pair C are labeled with VIC™, and
probes specific for amplicons generated using the at least one primer pair D are labeled with FAM™.

30. The composition according to claim 19, wherein:
the probe specific for amplifying at least a portion of the human RNase P gene is labeled with JUN™.

31. A kit for determining the presence or absence of adenovirus, metapneumovirus, rhinovirus/enterovirus, and/or parainfluenza in a sample, comprising a composition of claim 17.

32. The kit according to claim 31, further comprising a primer pair E and, optionally, a probe that specifically amplify at least a portion of a human RNase P gene.

33. The kit according to claim 32, wherein the primer pair E comprises at least one forward and reverse primer pair sequence in Table 5 and, when present, the probe that specifically amplifies at least a portion of the human RNase P gene sequence comprises at least one probe sequence in Table 5.

34. The method according to claim 8, wherein the at least one primer pair set and/or the additional primer pair set comprise at least one primer pair having a forward and reverse primer pair sequence as provided in Table 6, Table 7, Table 8, Table 9, Table 10 or Table 11; and wherein at least one of the probes has a probe sequence provided in Table 6, Table 7, Table 8, Table 9, Table 10 or Table 11.


SEQUENCE LISTING (180 sequences)

SEQ ID NO: 1 - DNA, 21 bp, synthetic construct
  misc_feature 1..21: note = Synthetic
  source 1..21: mol_type = other DNA
accgtttggatcctcgaaagc

SEQ ID NO: 2 - DNA, 19 bp, synthetic construct
  misc_feature 1..19: note = Synthetic
  source 1..19: mol_type = other DNA
gccctgatttcccgtttcc

SEQ ID NO: 3 - DNA, 17 bp, synthetic construct
  misc_feature 1..17: note = Synthetic
  source 1..17: mol_type = other DNA
cctgcatgtccagattt

SEQ ID NO: 4 - DNA, 25 bp, synthetic construct
  misc_feature 1..25: note = Synthetic
  source 1..25: mol_type = other DNA
acaaggaccgcatgtattctttctt

SEQ ID NO: 5 - DNA, 25 bp, synthetic construct
  misc_feature 1..25: note = Synthetic
  source 1..25: mol_type = other DNA
ccacgaatcctgagttgttatgttg

SEQ ID NO: 6 - DNA, 18 bp, synthetic construct
  misc_feature 1..18: note = Synthetic
  source 1..18: mol_type = other DNA
ccatgagtaggcaagttg

SEQ ID NO: 7 - DNA, 16 bp, synthetic construct
  misc_feature 1..16: note = Synthetic
  source 1..16: mol_type = other DNA
cctcgatgatgccgca

SEQ ID NO: 8 - DNA, 21 bp, synthetic construct
  misc_feature 1..21: note = Synthetic
  source 1..21: mol_type = other DNA
aacttgtttcccagggtgaag

SEQ ID NO: 9 - DNA, 23 bp, synthetic construct
  misc_feature 1..23: note = Synthetic
  source 1..23: mol_type = other DNA
ccggcgatgtgcatgtaagacca

SEQ ID NO: 10 - DNA, 25 bp, synthetic construct
  misc_feature 1..25: note = Synthetic
  source 1..25: mol_type = other DNA
cctgcaccattcccagataattttc

SEQ ID NO: 11 - DNA, 25 bp, synthetic construct
  misc_feature 1..25: note = Synthetic
  source 1..25: mol_type = other DNA
acctgtttgtaatgtgtggtttgga

SEQ ID NO: 12 - DNA, 17 bp, synthetic construct
  misc_feature 1..17: note = Synthetic
  source 1..17: mol_type = other DNA
ttccagccttgaattat

SEQ ID NO: 13 - DNA, 19 bp, synthetic construct
  misc_feature 1..19: note = Synthetic
  source 1..19: mol_type = other DNA
ctcggaatgcccactacca

SEQ ID NO: 14 - DNA, 25 bp, synthetic construct
  misc_feature 1..25: note = Synthetic
  source 1..25: mol_type = other DNA
gtcgatgtgactgagaaggtttcat

SEQ ID NO: 15 - DNA, 18 bp, synthetic construct
  misc_feature 1..18: note = Synthetic
  source 1..18: mol_type = other DNA
cctgtagctcacatggct

SEQ ID NO: 16 - DNA, 25 bp, synthetic construct
  misc_feature 1..25: note = Synthetic
  source 1..25: mol_type = other DNA
tccccatatggctctgaatgaaatc

SEQ ID NO: 17 - DNA, 25 bp, synthetic construct
  misc_feature 1..25: note = Synthetic
  source 1..25: mol_type = other DNA
gcacagatgagtatggtgaagaaga

SEQ ID NO: 18 - DNA, 19 bp, synthetic construct
  misc_feature 1..19: note = Synthetic
  source 1..19: mol_type = other DNA
atggtgaacatcaatcatg

SEQ ID NO: 19 - DNA, 25 bp, synthetic construct
  misc_feature 1..25: note = Synthetic
  source 1..25: mol_type = other DNA
gtacctgacttcctttgacgagaaa

SEQ ID NO: 20 - DNA, 24 bp, synthetic construct
  misc_feature 1..24: note = Synthetic
  source 1..24: mol_type = other DNA
ccgatgcagacaacatagtaaagc

SEQ ID NO: 21 - DNA, 16 bp, synthetic construct
  misc_feature 1..16: note = Synthetic
  source 1..16: mol_type = other DNA
ctggcgcttgtacctc

SEQ ID NO: 22 - DNA, 23 bp, synthetic construct
  misc_feature 1..23: note = Synthetic
  source 1..23: mol_type = other DNA
cattgctcgatctcactgaagtg

SEQ ID NO: 23 - DNA, 21 bp, synthetic construct
  misc_feature 1..21: note = Synthetic
  source 1..21: mol_type = other DNA
cctgaggcgaaaagaggttca

SEQ ID NO: 24 - DNA, 22 bp, synthetic construct
  misc_feature 1..22: note = Synthetic
  source 1..22: mol_type = other DNA
cagtgtggaagagcttagccgc

SEQ ID NO: 25 - DNA, 19 bp, synthetic construct
  misc_feature 1..19: note = Synthetic
  source 1..19: mol_type = other DNA
actggaggctcccttcgta

SEQ ID NO: 26 - DNA, 22 bp, synthetic construct
  misc_feature 1..22: note = Synthetic
  source 1..22: mol_type = other DNA
gctccgagtaacgaatgctgtt

SEQ ID NO: 27 - DNA, 17 bp, synthetic construct
  misc_feature 1..17: note = Synthetic
  source 1..17: mol_type = other DNA
ctcccttccgtaggcgc

SEQ ID NO: 28 - DNA, 34 bp, synthetic construct
  misc_feature 1..34: note = Synthetic
  source 1..34: mol_type = other DNA
ctatctatataatataccttatagatggaatggt

SEQ ID NO: 29 - DNA, 18 bp, synthetic construct
  misc_feature 1..18: note = Synthetic
  source 1..18: mol_type = other DNA
cacacagcgcgcactttt

SEQ ID NO: 30 - DNA, 23 bp, synthetic construct
  misc_feature 1..23: note = Synthetic
  source 1..23: mol_type = other DNA
ccaacatgtaaatgaggtaattt

SEQ ID NO: 31 - DNA, 25 bp, synthetic construct
  misc_feature 1..25: note = Synthetic
  source 1..25: mol_type = other DNA
ctaaaatacaagcaaaccgctgact

SEQ ID NO: 32 - DNA, 25 bp, synthetic construct
  misc_feature 1..25: note = Synthetic
  source 1..25: mol_type = other DNA
ggaaggacaaatggatacgctgtag

SEQ ID NO: 33 - DNA, 20 bp, synthetic construct
  misc_feature 1..20: note = Synthetic
  source 1..20: mol_type = other DNA
ttagtgcaagaggttttatg

SEQ ID NO: 34 - DNA, 19 bp, synthetic construct
  misc_feature 1..19: note = Synthetic
  source 1..19: mol_type = other DNA
accaccgtcagtgaaaacg

SEQ ID NO: 35 - DNA, 18 bp, synthetic construct
  misc_feature 1..18: note = Synthetic
  source 1..18: mol_type = other DNA
ggcgtcagtaacggtcac

SEQ ID NO: 36 - DNA, 23 bp, synthetic construct
  misc_feature 1..23: note = Synthetic
  source 1..23: mol_type = other DNA
cctgctctcacagatcacgggac

SEQ ID NO: 37 - DNA, 20 bp, synthetic construct
  misc_feature 1..20: note = Synthetic
  source 1..20: mol_type = other DNA
gggttgttcccactcaaggg

SEQ ID NO: 38 - DNA, 25 bp, synthetic construct
  misc_feature 1..25: note = Synthetic
  source 1..25: mol_type = other DNA
ggaattaaaacaggcgtacaaaggt

SEQ ID NO: 39 - DNA, 21 bp, synthetic construct
  misc_feature 1..21: note = Synthetic
  source 1..21: mol_type = other DNA
accagagtactagccgccacg

SEQ ID NO: 40 - DNA, 29 bp, synthetic construct
  misc_feature 1..29: note = Synthetic
  source 1..29: mol_type = other DNA
attggtctggatctctagaaatgacattc

SEQ ID NO: 41 - DNA, 25 bp, synthetic construct
  misc_feature 1..25: note = Synthetic
  source 1..25: mol_type = other DNA
ctggaggtgtgtaacataggatcaa

SEQ ID NO: 42 - DNA, 18 bp, synthetic construct
  misc_feature 1..18: note = Synthetic
  source 1..18: mol_type = other DNA
ctgttgccataaaactac

SEQ ID NO: 43 - DNA, 22 bp, synthetic construct
  misc_feature 1..22: note = Synthetic
  source 1..22: mol_type = other DNA
ggtgtgaagagtctattgagct

SEQ ID NO: 44 - DNA, 19 bp, synthetic construct
  misc_feature 1..19: note = Synthetic
  source 1..19: mol_type = other DNA
gacacccaaagtagtcggt

SEQ ID NO: 45 - DNA, 16 bp, synthetic construct
  misc_feature 1..16: note = Synthetic
  source 1..16: mol_type = other DNA
ctccggcccctgaatg

SEQ ID NO: 46 - DNA, 13 bp, synthetic construct
  misc_feature 1..13: note = Synthetic
  source 1..13: mol_type = other DNA
ctgcgtggcggcc

SEQ ID NO: 47 - DNA, 20 bp, synthetic construct
  misc_feature 1..20: note = Synthetic
  source 1..20: mol_type = other DNA
cacggacacccaaagtagtc

SEQ ID NO: 48 - DNA, 16 bp, synthetic construct
  misc_feature 1..16: note = Synthetic
  source 1..16: mol_type = other DNA
ctccggcccctgaatg

SEQ ID NO: 49 - DNA, 14 bp, synthetic construct
  misc_feature 1..14: note = Synthetic
  source 1..14: mol_type = other DNA
gcgtggctgcctgc

SEQ ID NO: 50 - DNA, 20 bp, synthetic construct
  misc_feature 1..20: note = Synthetic
  source 1..20: mol_type = other DNA
acacggacacccaaagtagt

SEQ ID NO: 51 - DNA, 16 bp, synthetic construct
  misc_feature 1..16: note = Synthetic
  source 1..16: mol_type = other DNA
ctccggcccctgaatg

SEQ ID NO: 52 - DNA, 19 bp, synthetic construct
  misc_feature 1..19: note = Synthetic
  source 1..19: mol_type = other DNA
cctggagccttgtgtcaca

SEQ ID NO: 53 - DNA, 22 bp, synthetic construct
  misc_feature 1..22: note = Synthetic
  source 1..22: mol_type = other DNA
aacacggacacccaaagtagtc

SEQ ID NO: 54 - DNA, 20 bp, synthetic construct
  misc_feature 1..20: note = Synthetic
  source 1..20: mol_type = other DNA
aaggtcgtaatgagcaattc

SEQ ID NO: 55 - DNA, 22 bp, synthetic construct
  misc_feature 1..22: note = Synthetic
  source 1..22: mol_type = other DNA
cgcatgtgcttggttgtgattc

SEQ ID NO: 56 - DNA, 21 bp, synthetic construct
  misc_feature 1..21: note = Synthetic
  source 1..21: mol_type = other DNA
ggctccagggttaaggttagc

SEQ ID NO: 57 - DNA, 16 bp, synthetic construct
  misc_feature 1..16: note = Synthetic
  source 1..16: mol_type = other DNA
ctccggcccctgaatg

SEQ ID NO: 58 - DNA, 19 bp, synthetic construct
  misc_feature 1..19: note = Synthetic
  source 1..19: mol_type = other DNA
cctggagccttgtgtcaca

SEQ ID NO: 59 - DNA, 22 bp, synthetic construct
  misc_feature 1..22: note = Synthetic
  source 1..22: mol_type = other DNA
aacacggacacccaaagtagtc

SEQ ID NO: 60 - DNA, 18 bp, synthetic construct
  misc_feature 1..18: note = Synthetic
  source 1..18: mol_type = other DNA
cccggaattgctcattac

SEQ ID NO: 61 - DNA, 18 bp, synthetic construct
  misc_feature 1..18: note = Synthetic
  source 1..18: mol_type = other DNA
gcccctgaatgcggctaa

SEQ ID NO: 62 - DNA, 25 bp, synthetic construct
  misc_feature 1..25: note = Synthetic
  source 1..25: mol_type = other DNA
acgaccttatcatcactggtttgtg

SEQ ID NO: 63 - DNA, 18 bp, synthetic construct
  misc_feature 1..18: note = Synthetic
  source 1..18: mol_type = other DNA
ccttaaccctggagcctt

SEQ ID NO: 64 - DNA, 27 bp, synthetic construct
  misc_feature 1..27: note = Synthetic
  source 1..27: mol_type = other DNA
acaaatagcatccatacatgactcgaa

SEQ ID NO: 65 - DNA, 28 bp, synthetic construct
  misc_feature 1..28: note = Synthetic
  source 1..28: mol_type = other DNA
ccacaaaacattgttaaatagctgctct

SEQ ID NO: 66 - DNA, 17 bp, synthetic construct
  misc_feature 1..17: note = Synthetic
  source 1..17: mol_type = other DNA
cccactcaagagaaaag

SEQ ID NO: 67 - DNA, 25 bp, synthetic construct
  misc_feature 1..25: note = Synthetic
  source 1..25: mol_type = other DNA
cttgtgtgcatgtgacagaaatcaa

SEQ ID NO: 68 - DNA, 22 bp, synthetic construct
  misc_feature 1..22: note = Synthetic
  source 1..22: mol_type = other DNA
cattgaacaacccctccttcct

SEQ ID NO: 69 - DNA, 17 bp, synthetic construct
  misc_feature 1..17: note = Synthetic
  source 1..17: mol_type = other DNA
ctggcttggataatcac

SEQ ID NO: 70 - DNA, 29 bp, synthetic construct
  misc_feature 1..29: note = Synthetic
  source 1..29: mol_type = other DNA
tcagtttggtagatagggtctctaatgtt

SEQ ID NO: 71 - DNA, 24 bp, synthetic construct
  misc_feature 1..24: note = Synthetic
  source 1..24: mol_type = other DNA
cacaggcccttgaaataacacttc

SEQ ID NO: 72 - DNA, 20 bp, synthetic construct
  misc_feature 1..20: note = Synthetic
  source 1..20: mol_type = other DNA
atgatggcagtaactaattg

SEQ ID NO: 73 - DNA, 28 bp, synthetic construct
  misc_feature 1..28: note = Synthetic
  source 1..28: mol_type = other DNA
tgaaatcagaacaaattctacccaggtt

SEQ ID NO: 74 - DNA, 22 bp, synthetic construct
  misc_feature 1..22: note = Synthetic
  source 1..22: mol_type = other DNA
aaagtgttgtgcagcctagaca

SEQ ID NO: 75 - DNA, 24 bp, synthetic construct
  misc_feature 1..24: note = Synthetic
  source 1..24: mol_type = other DNA
cagtgttttagttatttaaaaacc

SEQ ID NO: 76 - DNA, 29 bp, synthetic construct
  misc_feature 1..29: note = Synthetic
  source 1..29: mol_type = other DNA
tgggttaactgaacaattatcagtgttga

SEQ ID NO: 77 - DNA, 26 bp, synthetic construct
  misc_feature 1..26: note = Synthetic
  source 1..26: mol_type = other DNA
tttatgcttttcaaggtgtcccctaa

SEQ ID NO: 78 - DNA, 23 bp, synthetic construct
  misc_feature 1..23: note = Synthetic
  source 1..23: mol_type = other DNA
tagttccaagaactctagatctg

SEQ ID NO: 79 - DNA, 25 bp, synthetic construct
  misc_feature 1..25: note = Synthetic
  source 1..25: mol_type = other DNA
gagaaggcaaaagagctcaacaaaa

SEQ ID NO: 80 - DNA, 25 bp, synthetic construct
  misc_feature 1..25: note = Synthetic
  source 1..25: mol_type = other DNA
ccctgattgttgtcctgagtttctt

SEQ ID NO: 81 - DNA, 19 bp, synthetic construct
  misc_feature 1..19: note = Synthetic
  source 1..19: mol_type = other DNA
caagcggtgaatcagaaga

SEQ ID NO: 82 - DNA, 33 bp, synthetic construct
  misc_feature 1..33: note = Synthetic
  source 1..33: mol_type = other DNA
gccttattatctatagaaaagaatgaacgtgct

SEQ ID NO: 83 - DNA, 25 bp, synthetic construct
  misc_feature 1..25: note = Synthetic
  source 1..25: mol_type = other DNA
cttagcttgtcgttctgatcctact

SEQ ID NO: 84 - DNA, 19 bp, synthetic construct
  misc_feature 1..19: note = Synthetic
  source 1..19: mol_type = other DNA
caacactaatgagagaccc

SEQ ID NO: 85 - DNA, 24 bp, synthetic construct
  misc_feature 1..24: note = Synthetic
  source 1..24: mol_type = other DNA
tttgggacaaatcataatgtctcg

SEQ ID NO: 86 - DNA, 23 bp, synthetic construct
  misc_feature 1..23: note = Synthetic
  source 1..23: mol_type = other DNA
gccaactccagtaattgtggtta

SEQ ID NO: 87 - DNA, 23 bp, synthetic construct
  misc_feature 1..23: note = Synthetic
  source 1..23: mol_type = other DNA
ttgccccgcacttcatatttgca

SEQ ID NO: 88 - DNA, 26 bp, synthetic construct
  misc_feature 1..26: note = Synthetic
  source 1..26: mol_type = other DNA
agcttttaagaaacacagataaggct

SEQ ID NO: 89 - DNA, 24 bp, synthetic construct
  misc_feature 1..24: note = Synthetic
  source 1..24: mol_type = other DNA
catcaatgtacccttgaaccacat

SEQ ID NO: 90 - DNA, 27 bp, synthetic construct
  misc_feature 1..27: note = Synthetic
  source 1..27: mol_type = other DNA
cctgctcctgatattattgacaaacca

SEQ ID NO: 91 - DNA, 24 bp, synthetic construct
  misc_feature 1..24: note = Synthetic
  source 1..24: mol_type = other DNA
tctcttcaagggattcacctgagt

SEQ ID NO: 92 - DNA, 25 bp, synthetic construct
  misc_feature 1..25: note = Synthetic
  source 1..25: mol_type = other DNA
tgttgttgtgcctacatctctcttt

SEQ ID NO: 93 - DNA, 22 bp, synthetic construct
  misc_feature 1..22: note = Synthetic
  source 1..22: mol_type = other DNA
actgagactctttcaatatagc

SEQ ID NO: 94 - DNA, 25 bp, synthetic construct
  misc_feature 1..25: note = Synthetic
  source 1..25: mol_type = other DNA
gcaatcagccatgtggatattctgt

SEQ ID NO: 95 - DNA, 21 bp, synthetic construct
  misc_feature 1..21: note = Synthetic
  source 1..21: mol_type = other DNA
tccaacagcctgaggatctct

SEQ ID NO: 96 - DNA, 18 bp, synthetic construct
  misc_feature 1..18: note = Synthetic
  source 1..18: mol_type = other DNA
cagtgtagcacgctcatt

SEQ ID NO: 97 - DNA, 26 bp, synthetic construct
  misc_feature 1..26: note = Synthetic
  source 1..26: mol_type = other DNA
tgtctcttcaagggattcacctaagt

SEQ ID NO: 98 - DNA, 21 bp, synthetic construct
  misc_feature 1..21: note = Synthetic
  source 1..21: mol_type = other DNA
tgcagttgtggtgcctacatc

SEQ ID NO: 99 - DNA, 26 bp, synthetic construct
  misc_feature 1..26: note = Synthetic
  source 1..26: mol_type = other DNA
tctgtcatataaacatgctatattaa

SEQ ID NO: 100 - DNA, 25 bp, synthetic construct
  misc_feature 1..25: note = Synthetic
  source 1..25: mol_type = other DNA
actgtgaaacaagaggtgatcatgt

SEQ ID NO: 101 - DNA, 22 bp, synthetic construct
  misc_feature 1..22: note = Synthetic
  source 1..22: mol_type = other DNA
cattctctcgactgctcagcaa

SEQ ID NO: 102 - DNA, 16 bp, synthetic construct
  misc_feature 1..16: note = Synthetic
  source 1..16: mol_type = other DNA
cagcagcagggatcaa

SEQ ID NO: 103 - DNA, 29 bp, synthetic construct
  misc_feature 1..29: note = Synthetic
  source 1..29: mol_type = other DNA
gggaagctaacaagaaattacatgatact

SEQ ID NO: 104 - DNA, 30 bp, synthetic construct
  misc_feature 1..30: note = Synthetic
  source 1..30: mol_type = other DNA
tcagactaactttacatccagtagaactga

SEQ ID NO: 105 - DNA, 17 bp, synthetic construct
  misc_feature 1..17: note = Synthetic
  source 1..17: mol_type = other DNA
ccatggcagcatgttaa

SEQ ID NO: 106 - DNA, 30 bp, synthetic construct
  misc_feature 1..30: note = Synthetic
  source 1..30: mol_type = other DNA
acagagaaagcaaaagagcttaacaaaatt

SEQ ID NO: 107 - DNA, 31 bp, synthetic construct
  misc_feature 1..31: note = Synthetic
  source 1..31: mol_type = other DNA
tcttgactattgtcttgtatgtcttttggtt

SEQ ID NO: 108 - DNA, 20 bp, synthetic construct
  misc_feature 1..20: note = Synthetic
  source 1..20: mol_type = other DNA
tcttcttcttcagattctcc

SEQ ID NO: 109 - DNA, 22 bp, synthetic construct
  misc_feature 1..22: note = Synthetic
  source 1..22: mol_type = other DNA
ggaagaagcatggaaactagca

SEQ ID NO: 110 - DNA, 20 bp, synthetic construct
  misc_feature 1..20: note = Synthetic
  source 1..20: mol_type = other DNA
tcttcaagctctggacttgg

SEQ ID NO: 111 - DNA, 35 bp, synthetic construct
  misc_feature 1..35: note = Synthetic
  source 1..35: mol_type = other DNA
atgacatcagttattcttgtactatgtgtgctgct

SEQ ID NO: 112 - DNA, 20 bp, synthetic construct
  misc_feature 1..20: note = Synthetic
  source 1..20: mol_type = other DNA
tggacttggaacaagatggc

SEQ ID NO: 113 - DNA, 18 bp, synthetic construct
  misc_feature 1..18: note = Synthetic
  source 1..18: mol_type = other DNA
acttggccatgggtccta

SEQ ID NO: 114 - DNA, 24 bp, synthetic construct
  misc_feature 1..24: note = Synthetic
  source 1..24: mol_type = other DNA
atgctgcaagagccaatgtaggcc

SEQ ID NO: 115 - DNA, 26 bp, synthetic construct
  misc_feature 1..26: note = Synthetic
  source 1..26: mol_type = other DNA
caagacacaaaagacaaatggaaagg

SEQ ID NO: 116 - DNA, 25 bp, synthetic construct
  misc_feature 1..25: note = Synthetic
  source 1..25: mol_type = other DNA
gattgaattacaccaagattctgca

SEQ ID NO: 117 - DNA, 27 bp, synthetic construct
  misc_feature 1..27: note = Synthetic
  source 1..27: mol_type = other DNA
tcgattgctctcttctgtatccttcct

SEQ ID NO: 118 - DNA, 24 bp, synthetic construct
  misc_feature 1..24: note = Synthetic
  source 1..24: mol_type = other DNA
acagatgaacgaagaattgcatca

SEQ ID NO: 119 - DNA, 29 bp, synthetic construct
  misc_feature 1..29: note = Synthetic
  source 1..29: mol_type = other DNA
tcattatcaccaaaagcccaaatatatac

SEQ ID NO: 120 - DNA, 20 bp, synthetic construct
  misc_feature 1..20: note = Synthetic
  source 1..20: mol_type = other DNA
caaataccaggtgcttctca

SEQ ID NO: 121 - DNA, 25 bp, synthetic construct
  misc_feature 1..25: note = Synthetic
  source 1..25: mol_type = other DNA
ctgtggtacaaaacaaagctatgca

SEQ ID NO: 122 - DNA, 25 bp, synthetic construct
  misc_feature 1..25: note = Synthetic
  source 1..25: mol_type = other DNA
cagcttgtccaagtaggaacatttc

SEQ ID NO: 123 - DNA, 20 bp, synthetic construct
  misc_feature 1..20: note = Synthetic
  source 1..20: mol_type = other DNA
aactggaagaacatatttgg

SEQ ID NO: 124 - DNA, 27 bp, synthetic construct
  misc_feature 1..27: note = Synthetic
  source 1..27: mol_type = other DNA
aaacttaatcactcaaggatgtgcaga

SEQ ID NO: 125 - DNA, 28 bp, synthetic construct
  misc_feature 1..28: note = Synthetic
  source 1..28: mol_type = other DNA
ccgggtttaaatcaggatacatatctga

SEQ ID NO: 126 - DNA, 19 bp, synthetic construct
  misc_feature 1..19: note = Synthetic
  source 1..19: mol_type = other DNA
aaacctgatatgacttccc

SEQ ID NO: 127 - DNA, 22 bp, synthetic construct
  misc_feature 1..22: note = Synthetic
  source 1..22: mol_type = other DNA
ctgacacagcttgatccactca

SEQ ID NO: 128 - DNA, 29 bp, synthetic construct
  misc_feature 1..29: note = Synthetic
  source 1..29: mol_type = other DNA
aatcaatgtgcatattccaagaattgtgg

SEQ ID NO: 129 - DNA, 21 bp, synthetic construct
  misc_feature 1..21: note = Synthetic
  source 1..21: mol_type = other DNA
cagttcggaaaatgattctac

SEQ ID NO: 130 - DNA, 23 bp, synthetic construct
  misc_feature 1..23: note = Synthetic
  source 1..23: mol_type = other DNA
cattatggaatggaaacggtgca

SEQ ID NO: 131 - DNA, 27 bp, synthetic construct
  misc_feature 1..27: note = Synthetic
  source 1..27: mol_type = other DNA
gatgctttctctgctcctactttatgt

SEQ ID NO: 132 - DNA, 21 bp, synthetic construct
  misc_feature 1..21: note = Synthetic
  source 1..21: mol_type = other DNA
actgcatcctttgttgataag

SEQ ID NO: 133 - DNA, 28 bp, synthetic construct
  misc_feature 1..28: note = Synthetic
  source 1..28: mol_type = other DNA
tgtagacaatttagcactgatcctaggt

SEQ ID NO: 134 - DNA, 25 bp, synthetic construct
  misc_feature 1..25: note = Synthetic
  source 1..25: mol_type = other DNA
tggatctctcagctgtaacttagca

SEQ ID NO: 135 - DNA, 23 bp, synthetic construct
  misc_feature 1..23: note = Synthetic
  source 1..23: mol_type = other DNA
aagttagcaataattgaataaac

SEQ ID NO: 136 - DNA, 30 bp, synthetic construct
  misc_feature 1..30: note = Synthetic
  source 1..30: mol_type = other DNA
catgaagattacttgttctgaaaacagtcc

SEQ ID NO: 137 - DNA, 22 bp, synthetic construct
  misc_feature 1..22: note = Synthetic
  source 1..22: mol_type = other DNA
attgcagaggtgaatccacaca

SEQ ID NO: 138 - DNA, 19 bp, synthetic construct
  misc_feature 1..19: note = Synthetic
  source 1..19: mol_type = other DNA
atgcttttggaccattttc

SEQ ID NO: 139 - DNA, 25 bp, synthetic construct
  misc_feature 1..25: note = Synthetic
  source 1..25: mol_type = other DNA
cagggaaagattcttggagcttaga

SEQ ID NO: 140 - DNA, 23 bp, synthetic construct
  misc_feature 1..23: note = Synthetic
  source 1..23: mol_type = other DNA
tgcaacaaaggagcccatgataa

SEQ ID NO: 141 - DNA, 22 bp, synthetic construct
  misc_feature 1..22: note = Synthetic
  source 1..22: mol_type = other DNA
tctagcaaggaatttaagaaag

SEQ ID NO: 142 - DNA, 25 bp, synthetic construct
  misc_feature 1..25: note = Synthetic
  source 1..25: mol_type = other DNA
cgaagcagcaacattccagatgata

SEQ ID NO: 143 - DNA, 24 bp, synthetic construct
  misc_feature 1..24: note = Synthetic
  source 1..24: mol_type = other DNA
gtgtgattatgactgggctgttga

SEQ ID NO: 144 - DNA, 24 bp, synthetic construct
  misc_feature 1..24: note = Synthetic
  source 1..24: mol_type = other DNA
cttggagatctagataattaaaac

SEQ ID NO: 145 - DNA, 19 bp, synthetic construct
  misc_feature 1..19: note = Synthetic
  source 1..19: mol_type = other DNA
agatttggacctgcgagcg

SEQ ID NO: 146 - DNA, 20 bp, synthetic construct
  misc_feature 1..20: note = Synthetic
  source 1..20: mol_type = other DNA
gagcggctgtctccacaagt

SEQ ID NO: 147 - DNA, 23 bp, synthetic construct
  misc_feature 1..23: note = Synthetic
  source 1..23: mol_type = other DNA
ttctgacctgaaggctctgcgcg

SEQ ID NO: 148 - DNA, 25 bp, synthetic construct
  misc_feature 1..25: note = Synthetic
  source 1..25: mol_type = other DNA
ggacaggcttaaagctattggactt

SEQ ID NO: 149 - DNA, 25 bp, synthetic construct
  misc_feature 1..25: note = Synthetic
  source 1..25: mol_type = other DNA
gctgcttagcatctgttttcttctc

SEQ ID NO: 150 - DNA, 16 bp, synthetic construct
  misc_feature 1..16: note = Synthetic
  source 1..16: mol_type = other DNA
aagcagagagaaatgc

SEQ ID NO: 151 - DNA, 26 bp, synthetic construct
  misc_feature 1..26: note = Synthetic
  source 1..26: mol_type = other DNA
agctttttcatattgcttgcacacat

SEQ ID NO: 152 - DNA, 26 bp, synthetic construct
  misc_feature 1..26: note = Synthetic
  source 1..26: mol_type = other DNA
cgccacattaataggaggtcttttga

SEQ ID NO: 153 - DNA, 22 bp, synthetic construct
  misc_feature 1..22: note = Synthetic
  source 1..22: mol_type = other DNA
atgcagactaaatccacatcta

SEQ ID NO: 154 - DNA, 25 bp, synthetic construct
  misc_feature 1..25: note = Synthetic
  source 1..25: mol_type = other DNA
accttggctattcagttgttgctat

SEQ ID NO: 155 - DNA, 24 bp, synthetic construct
  misc_feature 1..24: note = Synthetic
  source 1..24: mol_type = other DNA
tggcaaagttgtgaagagttcaga

SEQ ID NO: 156 - DNA, 17 bp, synthetic construct
  misc_feature 1..17: note = Synthetic
  source 1..17: mol_type = other DNA
ctggtttttcaatttcc

SEQ ID NO: 157 - DNA, 27 bp, synthetic construct
  misc_feature 1..27: note = Synthetic
  source 1..27: mol_type = other DNA
ctggaaaacaatcttcatctccttctg

SEQ ID NO: 158 - DNA, 18 bp, synthetic construct
  misc_feature 1..18: note = Synthetic
  source 1..18: mol_type = other DNA
cgagcagcgcttctccat

SEQ ID NO: 159 - DNA, 17 bp, synthetic construct
  misc_feature 1..17: note = Synthetic
  source 1..17: mol_type = other DNA
acagtgaagaaacctcg

SEQ ID NO: 160 - DNA, 20 bp, synthetic construct
  misc_feature 1..20: note = Synthetic
  source 1..20: mol_type = other DNA
gagcgggttctgacctgaag

SEQ ID NO: 161 - DNA, 23 bp, synthetic construct
  misc_feature 1..23: note = Synthetic
  source 1..23: mol_type = other DNA
agcaacaactgaatagccaaggt

SEQ ID NO: 162 - DNA, 17 bp, synthetic construct
  misc_feature 1..17: note = Synthetic
  source 1..17: mol_type = other DNA
ctgtctccacaagtccg

SEQ ID NO: 163 - DNA, 26 bp, synthetic construct
  misc_feature 1..26: note = Synthetic
  source 1..26: mol_type = other DNA
gcttttttacttttgggtggtttcct

SEQ ID NO: 164 - DNA, 23 bp, synthetic construct
  misc_feature 1..23: note = Synthetic
  source 1..23: mol_type = other DNA
tcggcaggcttctgtaacattac

SEQ ID NO: 165 - DNA, 20 bp, synthetic construct
  misc_feature 1..20: note = Synthetic
  source 1..20: mol_type = other DNA
tagggtttggaatcactttc

SEQ ID NO: 166 - DNA, 19 bp, synthetic construct
  misc_feature 1..19: note = Synthetic
  source 1..19: mol_type = other DNA
gctcctctgctggattgca

SEQ ID NO: 167 - DNA, 21 bp, synthetic construct
  misc_feature 1..21: note = Synthetic
  source 1..21: mol_type = other DNA
ggagaccgtggagaacagaag

SEQ ID NO: 168 - DNA, 21 bp, synthetic construct
  misc_feature 1..21: note = Synthetic
  source 1..21: mol_type = other DNA
caaagcgaaaacccccgccgg

SEQ ID NO: 169 - DNA, 29 bp, synthetic construct
  misc_feature 1..29: note = Synthetic
  source 1..29: mol_type = other DNA
ctctgttacagttatgcagactaaatcca

SEQ ID NO: 170 - DNA, 27 bp, synthetic construct
  misc_feature 1..27: note = Synthetic
  source 1..27: mol_type = other DNA
gttgttgcagtttttccaaagacagaa

SEQ ID NO: 171 - DNA, 21 bp, synthetic construct
  misc_feature 1..21: note = Synthetic
  source 1..21: mol_type = other DNA
catctaaatgtgtgcaagcaa

SEQ ID NO: 172 - DNA, 27 bp, synthetic construct
  misc_feature 1..27: note = Synthetic
  source 1..27: mol_type = other DNA
aaaaccagtagctgtttctgaactctt

SEQ ID NO: 173 - DNA, 25 bp, synthetic construct
  misc_feature 1..25: note = Synthetic
  source 1..25: mol_type = other DNA
cagtgagatggatccgagacaataa

SEQ ID NO: 174 - DNA, 18 bp, synthetic construct
  misc_feature 1..18: note = Synthetic
  source 1..18: mol_type = other DNA
cacaactttgccaattgt

SEQ ID NO: 175 - DNA, 19 bp, synthetic construct
  misc_feature 1..19: note = Synthetic
  source 1..19: mol_type = other DNA
gccgctcaccttggctatt

SEQ ID NO: 176 - DNA, 26 bp, synthetic construct
  misc_feature 1..26: note = Synthetic
  source 1..26: mol_type = other DNA
gaagagttcagaaacagctactggtt

SEQ ID NO: 177 - DNA, 20 bp, synthetic construct
  misc_feature 1..20: note = Synthetic
  source 1..20: mol_type = other DNA
cagttgttgctatcaatcat

SEQ ID NO: 178 - DNA, 22 bp, synthetic construct
  misc_feature 1..22: note = Synthetic
  source 1..22: mol_type = other DNA
cttctgatggccgaggtttctt

SEQ ID NO: 179 - DNA, 18 bp, synthetic construct
  misc_feature 1..18: note = Synthetic
  source 1..18: mol_type = other DNA
tgcggtgtccaccaactg

SEQ ID NO: 180 - DNA, 20 bp, synthetic construct
  misc_feature 1..20: note = Synthetic
  source 1..20: mol_type = other DNA
ccatggagaaactagaaaaa